SEQUENCE LISTING (6 sequences)

SEQ ID NO: 1 - protein, 8 aa, artificial sequence
  binding to Scr kinase
RKGQRDLY

SEQ ID NO: 2 - protein, 8 aa, artificial sequence
  mutated binding region
AAGQRDLY

SEQ ID NO: 3 - protein, 5 aa, artificial sequence
  endoplasmatic reticulum retention signal
NQRRI

SEQ ID NO: 4 - protein, 58 aa, artificial sequence
  murine derived pep
YWSKNRKAKAKPVTRGTGAGARPRGQNKERPPPVPNPDYEPIRKGQRDLYSGLNQRAV

SEQ ID NO: 5 - protein, 18 aa, artificial sequence
  murine derived pep
DREYDQYSHLQGNQLRKK

SEQ ID NO: 6 - protein, 47 aa, artificial sequence
  murine derived pep
GHETGRPSGAAEVQALLKNEQLYQPLRDREDTQYSRLGGNWPRNKKS

(19) United States
(12) Patent Application Publication
(10) Pub. No.: US 2023/0070126 A1
(43) Pub. Date: Mar. 9, 2023

(54) CHIMERIC ANTIGEN RECEPTORS, VECTORS CODING FOR SUCH RECEPTORS AND THEIR USE IN THE MODIFICATION OF T CELLS

(71) Applicant: Albert-Ludwigs-Universitaet Freiburg, Freiburg (DE)

(72) Inventors: Wolfgang SCHAMEL, Freiburg (DE); Susana MINGUET, Freiburg (DE); Frederike HARTL, Rust (DE)

(73) Assignee: Albert-Ludwigs-Universitaet Freiburg, Freiburg (DE)

(21) Appl. No.: 17/599,084

(22) PCT Filed: Mar. 24, 2020
(86) PCT No.: PCT/EP2020/058075

§ 371 Date: Sep. 28, 2021

(30) Foreign Application Priority Data
Mar. 28, 2019 (EP) 19165672.7

(51) Int. Cl. C07K 16/28; C12N 15/86; C12N 15/62; C12N 5/0783; C07K 14/725; A61P 35/00

(52) U.S. Cl. CPC C07K 16/2803; C12N 15/86; C12N 15/625; C12N 5/0636; C07K 14/7051; A61P 35/00; C12N 2740/15043; A61K 39/00

(57) ABSTRACT

The present invention relates to a chimeric antigen receptor comprising at least the following components:
a) a peptidic structure capable of binding to a ligand;
b) an extracellular spacing structure;
c) a transmembrane domain;
d) none or at least one co-stimulatory domain; and
e) at least a TCR-derived activatory domain
   whereby a second TCR-derived activatory domain comprises the amino acid sequence RKGQRDLY (SEQ ID NO:1), which is capable to bind the lymphocyte specific Src kinase in a phosphorylation independent manner and its use in vectors for transducing T cells which are useful in the treatment of diseases, in particular tumors.

### PRIORITY

This application corresponds to the U.S. National phase of International Application No. PCT/EP2020/058075 filed Mar. 24, 2020, which, in turn, claims priority to European Patent Application No. 19165672.7 filed Mar. 28, 2019, the contents of which are incorporated by reference herein in their entirety.

### SEQUENCE LISTING

The instant application contains a Sequence Listing that has been submitted in ASCII format via EFS-Web and is hereby incorporated by reference in its entirety. Said ASCII copy, created on Sep. 28, 2021, is named LNK _233US_SEQ_LIST.txt and is 2,257 bytes in size.

### BACKGROUND OF THE INVENTION

Chimeric antigen receptors (CARs) are genetically constructed versatile synthetic receptors that provide T cells with a new and desired specificity. There is a therapeutic potential of T cells modified with a chimeric antigen receptor for the treatment of cancer, in particular in the treatment of B-cell malignancies. Currently, different CARs formats are available, such as the two FDA-approved anti-CD19 CARs (https://www.fda.gov/BiologicsBloodVaccines/CellularGeneTherapyProducts/ApprovedProducts/ucm573706.htm and http://www.fda.gov/NewsEvents/Newsroom/PressAnnouncements/ucm581216.htm), and more complex platforms such as the TRuC™ Platform (https://www.tcr2.com/our-approach/), bispecific chimeric antigen receptors or chimeric antigens using nanobodies (https://www.ncbi.nlm.nih.gov/pubmed/30672018).

The primary mediator of T cell activation is the T cell receptor (TCR). By replacing the naturally occurring antigen binding site with another binding site specific for a desired antigen a chimeric TCR can be generated. However, chimeric TCR must still recognize the tumor antigens associated to the major histocompatibility complex on the surface of tumor cells. This complex is often repressed by malignancies as mechanism of tumor scape. CARs, in contrast, recognize full proteins on the surface of tumor cells independently of the major histocompatibility complex. Human T cells, preferably CD4⁺ and/or in particular CD8⁺ T cells, can be genetically modified via transfection with a suitable vector coding for a CAR to redirect T cell specificity to the desired targets, e.g. proteins that are expressed in tumor cells.

Chang et al. (Trends in Molecular Medicine, May 2017, vol. 23, no. 5, pp 430-450) have described CARs as synthetic immuno-receptors for the treatment of cancer. Several structures of such chimeric antigen receptors are described and meanwhile several generations of CARs are known. The so-called first generation CARs consist of an extracellular antigen binding domain, an extracellular spacer, a transmembrane domain and a T cell activation (or activatory) domain, which normally is obtained from the CD3ζ peptide chain. CD3ζ is a subunit of the T cell receptor (TCR) complex that must be phosphorylated to initiate T cell activation signaling networks. The second generation of CARs contains, in addition to the components described for the first generation CARs, a co-stimulatory domain from co-stimulatory receptors of T cells, such as 41BB or CD28. The third generation of CARs contains, in addition to the components of the second generation CARs, a second co-stimulatory domain.

WO 2017/062820 discloses chimeric antigen receptors comprising a huge variety of different structures. The intracellular domain may be derived from many different sequences. There is no specific disclosure to the particular constructs of the present invention, since the constructs comprise a co-stimulatory domain followed by an activation domain but not a second activation domain.

WO 2018/136570 discloses chimeric antigen receptors against Axl which is a receptor tyrosine kinase in the tyro3-family of kinases or Ror2 which is also called receptor tyrosine kinase-like orphan receptor 2. Several ITAM motifs are disclosed therein, however, a construct according to the present invention having at least two activation domains is not disclosed.

It is one object of the present invention to provide alternative CAR designs, which have advantageous properties when used in the transfection of T cells. To date, most CAR designs have been overexpressed in the transfected T cells to obtain an efficient elimination of the tumor cells. However, overexpression of such CAR designs may have undesired properties as consequence of activation independently of target binding and/or non-physiological signal strengths (too much signal). These undesired properties included toxic side effects (cytokine release syndrome, CRS, as consequence of massive uncontrolled T cell activation) and/or inactivation of the transfected T cells by a process known as T cell exhaustion. Thus, an alternative strategy is, therefore, to specifically regulate the signaling properties of the CARs to make them more efficient.

### SUMMARY OF THE PRESENT INVENTION

The present invention discloses chimeric antigen receptors (CAR) which aims to improve the functionality comprising at least the following components:
a) a peptidic structure capable of binding to a ligand;
b) an extracellular spacing structure;
c) a transmembrane domain;
d) none or at least one co-stimulatory domain; and
e) at least one activation domain and
f) a second TCR-derived activation (or activatory) domain which includes the amino acid sequence RKGQRDLY (SEQ ID NO:1), and, in preferred embodiments, excludes the amino acid sequence NQRRI (endoplasmic reticulum retention signal, SEQ ID NO:3). The amino acid sequence RKGQRDLY (SEQ ID NO:1) is capable to bind the lymphocyte specific Src kinase.

The first activation domain (feature e)) is preferably derived from the T cell receptor (TCR). This domain comprises a cytoplasmic immune receptor tyrosine-based activation motif (ITAM), which is phosphorylated by a SCR tyrosine kinase and may therefore primarily activate the receptor.

The second activation domain (feature f)) comprises a sequence that binds a SCR tyrosine kinase (such as LCK) in a phosphorylated independent manner. This activation domain comprises SEQ ID NO:1 that binds a SCR tyrosine kinase (such as LCK) prior to phosphorylation of the cytoplasmic immune receptor tyrosine-based activation motifs (ITAMs).

In a preferred embodiment of the invention, the relevant sequence (SEQ ID NO:1) might be present not only once but twice or even three times. This makes the construct much more efficient.

The TCR-derived activation (or activatory) domain (feature e)) can be defined as a cytoplasmic sequence that is phosphorylated by a tyrosine kinase and activates therefore the CAR. The cytoplasmic signaling sequences within the cytoplasmic signaling portion of the CAR may be linked to each other in random or specified order. Both covalent and non-covalent bonds may be used to link the components.

### BRIEF DESCRIPTION OF THE FIGURES

FIG. 1 shows schematically a chimeric antigen receptor (CAR), which received FDA approval (prior art) (https://www.novartis.com/news/media-releases/kymriahr-tisageniecleucel-first-class-car-t-therapy-from-novartis-receives-second-fda-approval-treat-approopriate-rr-patients-large-b-cell-lymphoma). This CAR has the following components: An antiCD19sc part as an antigen binding part of the chimeric receptor; a linker derived from CD8 designated as CD8stalk; a transmembrane portion designated as CD8TM; a signal transducing unit designated as 41BB and the ζ part which is the ζ cytoplasmic tail taken from the CAR (Imai et al., 2004; Maude et al., 2018). This CAR is named in the present invention ζCAR.

FIG. 2 shows schematically embodiments of the present invention whereby each structure designated as εRKζCAR, εAAζCAR, miniεRKζCAR and miniεAAζCAR shows embodiments having the same components as shown in FIG. 1. In addition to the CAR shown in FIG. 1, the chimeric receptor designated as εRKζCAR contains a co-activatory domain derived from CD3ε (RK motif, sequence RKGQRDLY, SEQ ID NO:1). εAAζCAR corresponds to εRKζCAR whereby, however, the CD3ε motif is mutated which leads to lack of function (sequence AAGQRDLY, SEQ ID NO:2). The miniεRKζCAR is a modification of the εRKζCAR. miniεRKζCAR contains the CD3ε co-stimulatory domain (RK motif, sequence RKGQRDLY, SEQ ID NO:1) and lacks the_endoplasmatic reticulum retention signal having the amino acid sequence NQRRI (SEQ ID NO:3). The miniεAA ζCAR is a modification of miniεRKζCAR. miniεAAζCAR contains the non-functional CD3ε co-stimulatory domain (sequence AAGQRDLY, SEQ ID NO:2)_and lacks the endoplasmic reticulum retention signal having the amino acid sequence NQRRI (SEQ ID NO:3).

FIG. 3 schematically represents of the constructs used in the vectors for transfection. In the upper line with the designation ζCAR a construct according to the prior art is shown. After elongation factor 1α and a signal peptide, the antigen binding structure is represented by V_{L} and V_{H} coding for the FMC63 (amino acids 1-267, GenBank ID: HM852952.1) anti-CD19 region. Then the structures from the CAR ECD (extracellular domain)+TM (transmembrane domain) and ICD (intracellular domain) are present in all constructs. Furthermore, all constructs contain the ICD of ζ and the green fluorescent protein (GFP), which allows the identification of cells transfected with the constructs.

In the next lines, the constructs according to the present invention are shown wherein a fragment of the ICD from CD3ε is present (aa amino acids 153-202, GenBank ID: NM_000733.3). The constructs codified for the following chimeric receptors: εRKζCAR, εAAζCAR, miniεRKζCAR, miniεAA ζCAR εRK highlights the presence of the wild-type RK motif. εAA highlights that the RK motif is mutated (RK→AA). miniεRKζCAR and miniεAA ζCAR correspond to εRKζCAR, εAAζCAR, respectively; whereby, however, the endoplasmatic reticulum retention part has been deleted (sequence NQRRI, SEQ ID NO:3).

FIG. 4 shows the results of Experiment 1. Expanded healthy human T cells were transfected with lentiviral vectors coding for the prior art construct shown in FIG. 1 (ζCAR). A panel of different multiplicity of infection (MOI) was used to get different levels of CAR expression. εRKζCAR, εAAζCAR, miniεRKζCAR and miniεAAζCAR were also used in this experiment to a MOI of 5. The level of surface CAR expression was measured by a flow cytometric assay specifically detecting the antiCD19sc part with a fluorescent-labeled antibody. The level of surface CAR expression is proportional to the specific florescent detected and it is indicated as Mean fluorescence intensity (MFI). The results in FIG. 4 show that the surface CAR expression of the chimeric receptors according to the invention is reduced when compared to the prior art construct at the same multiplicity of infection (MOI of 5). The deletion of an endoplamatic reticulum retention signal (sequence NQRRI, SEQ ID NO:3) increases substantially the surface CAR expression. The results of two independent donors of healthy human T cells are shown.

FIG. 5 shows the results of Experiment 2. FIG. 5 shows the percent of specific killing of CD19-expressing cells (Nalm6 pre-B acute lymphoblastic leukemia) plotted against the surface CAR expression. These experiments show that the anti-tumor activity of cells expressing the embodiments εRKζCAR, εAAζCAR, miniεRKζCAR of the present invention was significantly higher than the anti-tumor activity of cells expressing the prior art ζCAR with the most similar surface levels. The anti-tumor activity of cells expressing the embodiment miniεAAζCAR of the present invention was equal or lower than the anti-tumor activity of cells expressing the prior art ζCAR with the most similar surface levels. These results demonstrated that loss-of-function mutation of the RK motif (sequence RKGQRDLY, SEQ ID NO:1 to sequence AAGQRDLY, SEQ ID NO:2) abolished the enhancement in tumor killing of the embodiments εRKζCAR and miniεRKζCAR of the present invention. The experiments were performed with expanded T cells from two different healthy donors.

FIG. 6 shows the results of Experiment 3. FIG. 6 shows the percent of specific killing of CD19-expressing cells (DAUDI cell line, which is derived from Burkitt-lymphoma) plotted against the surface CAR expression. Data processing was done as described in FIG. 5. Comparison of the CARs according to the invention to the prior art CAR (ζCAR) at the same level of surface expression demonstrates that the embodiments of the present invention εRKζCAR, εAAζCAR and miniεRKζCAR have a much higher anti-tumoral activity against DAUDI lymphoma cells. Loss-of-function mutation of the RK motif (sequence RKGQRDLY, SEQ ID NO:1 to sequence AAGQRDLY, SEQ ID NO:2) abolished the enhancement in tumor killing of the embodiments εRKζCAR and miniεRKζCAR of the present invention. The experiments were performed with expanded T cells from two different healthy donors.

FIG. 7 shows an alternative analysis of selected data derived from Experiment 2. The anti-tumor activity, or with other words the killing activity, of human T cells transduced with the lentiviral vector encoding for the prior art CAR (ζCAR) was compared with the killing activity of human T cells transduced with the vectors coding for εRKζCAR or εAAζCAR for the most similar level of expression. FIG. 7 shows clearly that the chimeric receptors of the present invention have substantially higher anti-tumor killing activity when normalized to the same level of expression. The target cell, which was used in this experiment was Nalm6 (pre-B acute lymphoblastic leukemia).

FIG. 8 pools the results of four healthy donors after independently done experiments performed as Experiment 2. In FIG. 8, enhanced anti-tumor activity is defined as the difference between the specific killing activities of T cells expressing εRKζCAR or εAAζCAR and of T cells expressing ζCAR with the most similar surface expression. Mean values+/−SEM and p values using unpaired Student's t test are shown. When the number of expressed chimeric receptors on the cell surface is brought to a comparative level, the T cells expressing the εRKζCAR receptors show a higher anti-tumoral activity than T cells expressing the εAAζCAR. These data indicate that mutation of the RK motif strongly reduces the enhancement in tumor killing. Altogether, introducing the RK motif in a well-established CAR seems to be a good approach to increase the anti-tumor activity in vitro when the number of expressed chimeric receptors on the cell surface is brought to a comparative level. The target cell used in these experiments was Nalm6 (pre-B acute lymphoblastic leukemia).

FIG. 9 shows an alternative analysis of selected data derived from Experiment 3. The anti-tumor activity of human T cells transduced with the lentiviral vector encoding for the prior art CAR (ζCAR) was compared with the killing activity of human T cells transduced with the vectors coding for εRKζCAR or εAAζCAR for the most similar level of expression using DAUDI (Burkitt-lymphoma) cells as targets. FIG. 9 shows clearly that the chimeric receptors of the present invention have substantially higher anti-tumor killing activity when normalized to the same level of expression.

FIG. 10 pools the results of five healthy donors after independently done experiments performed as Experiment 3. In FIG. 10, enhanced anti-tumor activity is defined as in FIG. 8. The target cell used in these experiments was DAUDI (Burkitt-lymphoma).

FIG. 11 shows an alternative analysis of selected data derived from Experiment 2. The anti-tumor killing activity of human T cells transduced with the lentiviral vector encoding for the prior art CAR (ζCAR) was compared with the killing activity of human T cells transduced with the vectors coding for miniεRKζCAR or miniεAAζCAR for the most similar level of expression. FIG. 11 shows clearly that only the chimeric receptors containing the RK motif (miniεRKζCAR) of the present invention have substantially higher anti-tumor killing activity when normalized to the same level of expression. These data indicate that mutation of the RK motif strongly reduces the enhancement in tumor killing of the miniεRKζCAR presented in this invention. The target cells used in this experiment were Nalm6 (pre-B acute lymphoblastic leukemia).

FIG. 12 pools the results of two healthy donors after independently done experiments performed as Experiment 2 and compares miniεRKζCAR with miniεAAζCAR to assay for the role of the RK motif. As above, enhanced anti-tumor activity is defined as the difference between the specific killing activities of T cells expressing miniεRKζCAR or miniεAAζCAR and of T cells expressing ζCAR with the most similar surface expression. When the number of expressed chimeric receptors on the cell surface is brought to a comparative level, the T cells expressing the miniεRKζCAR receptors show a significantly higher anti-tumoral activity than T cells expressing the miniεAAζCAR. These data indicate that mutation of the RK motif strongly reduces the enhancement in tumor killing. The target cell used in these experiments was Nalm6 (pre-B acute lymphoblastic leukemia).

FIG. 13 pools the results of two healthy donors after independently done experiments performed as Experiment 2 and compares miniεRKζCAR with miniεAAζCAR to assay for the role of the RK motif using DAUDI (Burkitt-lymphoma) as target cells. As above, enhanced anti-tumor activity is defined as the difference between the specific killing activities of T cells expressing miniεRKζCAR or miniεAAζCAR and of T cells expressing ζCAR with the most similar surface expression. When the number of expressed chimeric receptors on the cell surface is brought to a comparative level, the T cells expressing the miniεRKζCAR receptors show a significantly higher anti-tumoral activity than T cells expressing the miniεAAζCAR. These data indicate that mutation of the RK motif strongly reduces the enhancement in tumor killing.

FIG. 14 shows the results of Experiment 4. T cell exhaustion is a major factor limiting anti-tumor responses. For this reason, up-regulation of the three exhaustion markers TIM3, LAG3 and PD1 was checked upon expression of the ζCAR, miniεRKζCAR or miniεAAζCAR. Expression of ζCAR in T cells resulted in significant up-regulation of the three exhaustion markers TIM3, LAG3 and PD1 compared to mock transduced cells in the absence of any CD19-positive cells (p<0.0001). These results are consistent with previous studies linking CAR expression levels to ligand-independent signals. In contrast, miniεRKζCAR and miniεAAζCAR transduced cells remained exhausted as the mock cells (p>0.05).

FIG. 15 shows a schematic representation of the pre-clinical model applied to assay the anti-tumor activity of the four chimeric antigen receptors used in the present invention (FIG. 2) in comparison to the well-characterized prior art ζCAR (FIG. 1, Imai et al., 2004; Maude et al., 2018). The tumor cells used in this model were Nalm6 (pre-B acute lymphoblastic leukemia).

FIG. 16 plots the results of the Log-rank Mantel-Cox survival test of Nalm6-bearing mice treated 3 days after tumor cell inoculation with 3×10⁶ (left) or 1.5×10⁶ (right) ζCAR-expressing human T cells (n=4-8 mice). NT indicates non-transduced T cells. ** p<0.01. FIG. 16 corresponds to Experiment 5.

FIG. 17 plots the results of Log-rank Mantel-Cox survival test of Nalm6-bearing mice treated with 1.5×10⁶ CAR-expressing human T cells 3 days after tumor cell inoculation (n=6-12 mice pooled from two independently performed experiments). Mice were monitored by in vivo bioluminescence and the images shown are form one of the two experiments (right panel). NT indicates non-transduced control T cells; ζCAR indicates ζCAR-transduced T cells. miniεRKζCAR and miniεAAζCAR indicate miniεRKζCAR-transduced T cells and miniεAAζCAR-transduced T cells, respectively. ** p<0.01, *** p<0.001. FIG. 17 shows the results of Experiment 6.

FIG. 18 shows schematically a TRuC (prior art) (also named TFP or TRuC™ Platform, https://www.tcr2.com/our-approach/). This TRuC has the following components: An antiCD19sc part as an antigen binding part; a linker to bind the antigen binding part to the extracellular domain of CD3ε; the extracellular domain of CD3ε; the transmembrane domain of CD3ε; and the cytoplasmic tail taken from CD3ε.

FIG. 19 shows schematically embodiments of the present invention whereby the structure designated as 2xRKTRuC shows embodiments having the same components as shown in FIG. 1. In addition, the chimeric receptor designated as 2xRKTRuC contains a duplication of the activation domain derived from CD3ε. (RK motif, sequence RKGQRDLY, SEQ ID NO:1) in each CD3εchain (each TRuC has two CD3εchains).

FIG. 20 shows the results of Experiment 7. Expanded healthy human T cells were transfected with lentiviral vectors coding for the prior art construct shown in FIG. 18 (TRuC) or the 2xRKTRuC (FIG. 19). The level of surface CAR expression was measured by a flow cytometric assay specifically detecting the antiCD19sc part with a fluorescent-labeled antibody. The level of surface TRuC expression is proportional to the specific fluorescent detected and it is indicated as Mean fluorescence intensity (MFI). The results in FIG. 20 show that the surface TRuC expression of the chimeric receptor 2xRKTRuC according to the invention is similar to the prior art construct at the same multiplicity of infection (MOI of 5). The results of one donor of healthy human T cells are shown.

FIG. 21 shows the results of Experiment 8. FIG. 21 shows the percent of specific killing of CD19-expressing cells (Nalm6 pre-B acute lymphoblastic leukemia) at different times after contact with the primary T cells expressing the prior art TRuC or the novel 2xRKTRuC. These experiments show that the anti-tumor activity of cells expressing the embodiment 2xRKTRuC of the present invention was significantly higher than the anti-tumor activity of cells expressing the prior art TRuC at different times after contact of the tumor cells with the primary T cells expressing the indicated TRuC. The experiments were performed with expanded T cells from one healthy donor. FIG. 21 shows the superior effect obtainable by the novel 2xRKTRuC of the present invention compared with the prior art construct (TRuC, also named “TFP” or TFP platform).

FIG. 22 shows (A-C) In situ proximity ligation assay (PLA) of the TCR (CD3δ) and LCK. A TCR:LCK distance smaller than 80 nm results in a red fluorescent signal. Nuclei were stained with DAPI. (D) PLA of the TCR and LCK in purified primary human T cells at 37° C. for 5 min. (E) Purified human T cells were treated with PP2 and either left unstimulated, stimulated with anti-CD3ε (OKT3) or with perV at 4° C. for 120 min. Statistical analysis was performed using unpaired Student's t test. Mean values+/−SEM and p values are shown (ns: not significant).

FIG. 23 (A) shows the sequences (SEQ ID NO:4-6) of the murine CD3-derived peptides used in the study. FIG. 19 (B) shows a pull down (PD) assay using glutathione beads bound to GST (−) or to GST-fusion proteins containing either the LCK SH2 domain (SH2) or both LCK SH2 and SH3 domains (SH2-SH3) was performed. These beads were incubated with the indicated biotinylated peptides as shown in A (“PP” refers to doubly ITAM tyrosine phosphorylated peptides). Immunoblotting was performed using streptavidin-HRP and anti-GST antibodies (C) PD assays using GST-SH3(LCK) and GST-SH3(NCK) proteins as in B. FIG. 19 (D) shows Jurkat T cell lysates that were incubated with the biotinylated CD3ε-derived peptides and streptavidin-coupled beads. Immunoblotting was performed using the indicated antibodies. FIG. 19 (E) shows Jurkat T cells that were left untreated (−) or stimulated for 5 min at 37° C. with anti-CD3ε antibody (+). Lysates were incubated with SH3(LCK)-coupled beads in a pull-down assay (PD). Immunoblotting was done using anti-ζ and anti-GST antibodies. Mean values+/− SEM and p values are indicated.

FIG. 24 shows an NMR titration experiment showing the change in the ¹H, ¹⁵N HSQC spectrum of free ¹⁵N-labeled SH3(LCK) (black, 50 μM) upon addition of 8-(red) or 16-fold (green) molar excess of unlabeled GST-CD3εEcytoplasmic tail. sc: side chain. Black arrows connect signals coming from the same residue. Blue arrows indicate the direction of changes. Normalized chemical shift changes as a function of protein sequence (bottom panel).

FIG. 25 shows a proximity ligation assay (PLA) between the TCR (CD36δ) and LCK was performed as in Figure A. H8εsh cells expressing different CD3εvariants were treated with PP2 and either left unstimulated (uns), stimulated with an anti-Cd3ε antibody (145-2C11) or with pervanadate (perV) at 4° C. Statistical analysis was performed between antibody-stimulated cells (grey bars) using unpaired Student's t test on pooled data of four independent experiments. Mean values+/−SEM and p values are indicated. ns: not significant.

FIG. 26 shows a backbone representation of the modeled complex formed by SH3(LCK) with the CD3ε cytoplasmic tail based on the NMR data and the current literature is displayed. Green: SH3(LCK); brown: CD3ε. Interactions are shown as dotted lines: hydrogen bonds (green), salt bridges and electrostatic interactions (black), hydrophobic, π-sigma and π-alkyl bonds (purple).

FIG. 27 (A) shows a scheme depicting the reporter for LCK activity at the TCR (ζ-Reporter). ζ was fused to LCK enzymatically inactive kinase domain (K273A) using a flexible linker. FIG. 23 (B) shows the immunoprecipitation of the TCR from MA5.8 cells (murine T cells lacking ζ expression) reconstituted with or with the ζ-Reporter. Cells were either left untreated (−) or stimulated (+) at 37° C. with anti-Cd3ε (145-2C11). Immunoblotting was done using the indicated antibodies. FIG. 23 (C) shows the quantification of the data from three independent experiments performed as in B. The relative value of phosphorylation in stimulated samples was set to 100% for each experiment. Statistical analysis compared the basal phosphorylation state of ζY142 and of Y394 both within the ζ-Reporter using Student's t test. Mean values+/− SEM and p values are shown. FIG. 23 (D) shows the fold induction of Y394 phosphorylation or of the ζ ITAM tyrosine (pζY142) both within the ζ-Reporter was calculated from three experiments done as in B and statistical analysis was performed using Student's t test. Mean values+/−SEM and p values are shown.

FIG. 28 (A) shows the immunoprecipitation of phosphotyrosine-containing proteins using 4G10 (top panel). Cells were left unstimulated or stimulated with anti-Cd3ε (145-2C11) for 30 seconds. Immunoblotting was done using anti-Cd3c (M20ε) and anti-c. The signal of stimulated CD3ε WT-expressing cells was set to 100% for each independent experiment. Data from four independent experiments were pooled and antibody-stimulated samples were compared to WT using unpaired Student's t test. Mean+/−SEM and p values are shown. FIG. 24 (B) shows the flow cytometric analysis of calcium influx from the different H8εsh transfectants upon stimulation with 3 μg/m1 anti-Cd3ε (145-2C11). FIG. 24 (C) shows the IL2 production of the different H8εsh transfectants after 24 h stimulation with anti-Cd3ε (145-2C11) and anti-hCD28 was assayed by ELISA. The value of IL2 produced by CD3εWT-cells was set to 100% for each experiment. Data from five independent experiments were pooled and the statistical analysis compared H8εsh-CD3εWT with each of the H8εsh cell variants using unpaired Student's t test. Mean values+/−SEM and p values are shown.

### DETAILED DESCRIPTION OF THE PRESENT INVENTION

A chimeric antigen receptor (CAR) in the sense of the present invention is considered as an artificially constructed hybrid protein having a peptidic structure capable of binding to a ligand linked to a signaling sequence via a transmembrane domain. Characteristics of the

CARs according to the present invention include their ability to redirect T-cell specificity and reactivity towards a selected target in a non-MHC-restricted manner and exploiting the antigen-binding properties of any binding domain.

The regions of the CAR according to the invention are:

### Peptidic Structure Capable of Binding to a Ligand (Feature A)):

A decisive part for the specific function of the CAR is the peptidic structure capable of binding to a ligand. The purpose of this part of CAR is to bring the modified T cell in close connection to the target cell (tumor cell) whereby the peptidic structure can bind sufficiently specific and with sufficient strength to the target molecule. The target molecule should be a specific structure, which is expressed (ideally) only at the target cell, which should be destroyed. Such a ligand can be a specific tumor antigen. There are many different tumor antigens known like for example α-feto-protein, carcinoembryonic antigen, epithelial tumor antigen, PSA or PSMA to name only a few.

The selection of the antigen-binding domain depends on the particular type of disease to be treated. Tumor antigens are well-known in the art and encompass for example a glioma-associated antigen, carcinoembryonic antigen (CEA), beta-human chorionic gonadotropin, α-feto-protein (AFP) and many others.

In most cases, however, these tumor antigens are also expressed in subpopulations of healthy cells. It is important therefore, that the healthy cells that are going to be co-targeted are not vital for the patient.

The ligand to which the peptidic structure binds may also be a specific antigen expressed at the surface of cells infected with viruses, bacteria or other organisms which cause diseases in humans and which are difficult to treat, because the foreign disease causing agent lives and propagates with cells of the host. The ligand which the peptidic structures binds may also be a specific antigen of a hematopoietic disorder resulting in an autoimmune disease, such as for example neurological disorders, rheumatological disorders, hematological immunocytopenias, and gastrointestinal disorders (e.g. inflammatory bowel disease).

Methods to generate peptidic structures, which bind to such ligands are well-known. The probably most commonly known method is the generation of monoclonal antibodies with the help of mice. When a suitable monoclonal antibody has been identified, the amino acid sequences can be obtained. Such monoclonal antibodies or the antigen binding parts thereof may be genetically manipulated to obtain binding structures which exhibit a strong specific binding to the target sequence and which do preferably not contain foreign amino acid sequences which may induce undesired immunological effects. Therefore, such antigen-binding structures are preferably humanized when used as part of the CAR.

Particularly, useful examples of the extracellular element according to the present invention include extracellular elements derived from antibodies (H chain and L chain) and variable regions of a TCR (TCRα, TCRβ, TCRγ, TCRδ), CD8α, CD8β, CD11A, CD11B, CD11C, CD18, CD29, CD49A, CD49B, CD49D, CD49E, CD49F, CD61, CD41, and CD51. In some embodiments, the entire protein may be used effectively. In some embodiments, a domain capable of binding to an antigen or a ligand, for example, an extracellular domain of an antibody Fab fragment, an antibody variable region [V region of H chain (VH) and V region of L chain (VL)] or a receptor can be used.

Such peptidic sequence can for example be humanized. A well-known form of such peptidic structures are humanized scFv structures which are used in preferred embodiments of the present invention.

### Extracellular Spacing Structure (Feature B)):

The antigen binding part (such as scFv structures) and the transmembrane region are usually linked by a hinge region. The purpose of this hinge region is to bring the antigen-binding region in a suitable steric position and distance to the cell membrane.

The extracellular spacing structure is intended to bring the structure, which binds to the ligand in a correct sterical orientation apart from the cytoplasma membrane of the T cell. Such extracellular spacing structure may be derived from receptors of T cells or it may also be an artificial structure like a polyglycine linker having for example 5-10 amino acids or combinations thereof. Other structures and combinations of such structures are also useful.

### Transmembrane Domain (Feature C)):

The transmembrane domain is usually obtained from a T cell receptor and stretches through the cytoplasmic membrane. The transmembrane domain helps to embed the CAR into the cytoplasma membrane of the T cell.

The transmembrane domain may be derived either from a natural or from a synthetic source. Where the source is natural, the domain may be derived from any membrane-bound or transmembrane protein. Transmembrane regions of particular use in this invention may be derived from TCRalpha, TCRbeta, TCRgamma or TCRdelta, from the zeta chain (CD247) of the TCR, CD3 epsilon, CD3 gamma, CD3delta, CD28, CD45, CD4, CDS, CD8, CD9, CD16, CD22, CD33, CD37, CD64, CD50, CD56, CD134, CD137, CD154. Alternatively, the transmembrane domain may be derived from any member of the novel tumor necrosis factor receptor superfamily (TNFRSF) member transmembrane domain. The transmembrane domain may be synthetic, in which case it will comprise predominantly hydrophobic residues such as leucine and valine.

### None or at Least One Co-Stimulatory Domain (Feature D)):

The main function of a co-stimulatory domain is to provide additional signals to the T cell for optimal T cell activation. The CAR according to the present invention in a preferred embodiment contains one co-stimulatory domain, which is derived from the 41BB receptor. In particular, the 41BB co-stimulatory domain seems to prolong the persistence of CAR T cells in individuals upon injection, allowing a better elimination of the tumor cells.

A CAR can, according to the present invention, have none, one or more, such as two or three co-stimulatory domains. The CAR used in the present invention as proof of principle contains one co-stimulatory domain, which is derived from the 41BB receptor.

### The Activation Domain Which is Preferably TCR-Derived (Feature E)):

At least one TCR-derived activation (or activatory) domain may be defined as a cytoplasmic sequence that is phosphorylated by a tyrosine kinase and is therefore primarily responsible for the activation of the CAR.

The T cell antigen receptor (TCR) is a multimeric protein complex expressed on the surface of T cells. The αβ TCR is composed of the ligand-binding TCRαβ heterodimer and the signal-transducing CD3 complex. In contrast to TCRαβ , CD3 subunits contain immune-receptor tyrosine-based activation motifs (ITAMs) in their cytoplasmic tails that mediate signal transduction. Upon TCR binding to its ligand (major histocompatibility complex bound to an antigenic peptide, MHCp), the ITAMs are phosphorylated on tyrosine residues by the members of the sarcoma (SRC) family tyrosine kinase, namely LCK, and TCR signaling is initiated. Phosphorylation of the ITAMs is absolutely needed for T cell activation by the TCR. In the absence of this phosphorylation, T cells do not get activated.

ITAM phosphorylation is also crucial for signal initiation of CARs. Therefore, CARs contain at least one TCR-derived activation domain defined as a cytoplasmic sequence that is phosphorylated by a tyrosine kinase and therefore primarily activates the CAR.

As used herein, the term “TCR-derived activation domain” and the term “primary cytoplasmic signaling sequence” are used interchangeably. “Activation domain” and “activatory domain” are used interchangeably. As used herein, a “TCR-derived activation domain” is defined to be a sequence that regulates primary activation of T cells. This primary cytoplasmic signaling sequence may comprise a signal transduction motif known as an immuno-receptor tyrosine-based activation motif (ITAM).

Examples of ITAM containing primary cytoplasmic signaling sequences that are of particular use in the CARs disclosed herein include, but are not limited to those derived from TCR zeta (CD3 zeta or CD247), FcR gamma, FcR beta, CD3 gamma, CD3 delta, CD3 epsilon, CDS, CD22, CD79a, CD79b, and CD66d. Thus, a suitable activation domain includes the consensus ITAM domain (D/ExxD/ExxxxxxxYxxl/LxxxxxxxYxxl/L with “x” being a non-conserved amino acid: Reth, Nature, vol. 338, pp. 383-384, 1989) with at least 50%, 60%, 70%, 75%, 80%, 85%, 90%, 95%, 96%, 97%, 98%, 99% or 100% sequence identity to the original sequences of TCR zeta (CD3 Zeta or CD247), FcR gamma, FcR beta, CD3 gamma, CD3 delta, CD3 epsilon, CDS, CD22, CD79a, CD79b, and CD66d.

### Second Activation Domain (Feature F)):

The second activation domain comprises at least one sequence that binds a tyrosine kinase in a phospho-tyrosine-independent manner.

ITAM phosphorylation is also crucial for signal initiation of CARs. Therefore, CARs contain at least one TCR-derived activation domain (feature e)) defined as a cytoplasmic sequence that is phosphorylated by a tyrosine kinase and therefore primarily activates the CAR.

ITAMs are phosphorylated on tyrosine residues by the members of the sarcoma (SRC) family tyrosine kinase, namely Src, Yes, Fyn, Fgr, Lck, Hck, Blk, Lyn and Frk. All the members of the sarcoma (SRC) family tyrosine kinase contain six conserved domains: an N-terminal myristoylated segment, an SH2 domain, an SH3 domain, a linker region, a tyrosine kinase domain, and C-terminal tail. Both SH2 and SH3 domains are involved in cooperative, intramolecular interactions between the SH2 domain and phosphorylated Y505 and between the SH3 domain and a proline-rich sequence flanked by the SH2 and kinase domains. This autoinhibited kinase is a precariously set mousetrap, in which alternative intermolecular interactions can be sufficient for activation. SH3 domain displacement via binding to Proline-dependent sequences activates members of the sarcoma (SRC) family tyrosine kinase.

An important aspect of the invention is introducing a sequence (feature f)) in the cytoplasmic tail of the CAR that binds a tyrosine kinase in a phosphorylation-independent manner via its SH3 domain and thereby mediates the recruitment of the tyrosine kinase allowing the phosphorylation of the ITAMs. The disclosure herein is exemplified by the RKGQRDLY (SEQ ID NO:1) sequence from CD3ε.

The amino acid sequence RKGQRDLY (SEQ ID NO:1) is capable to bind the lymphocyte specific Src kinase (know as LCK). It should be emphasized that this sequence is contained in the second activatory domain (feature f)).

According to the present invention, the CAR has two TCR-derived activation domains derived from a T cell receptor (TCR (features e) and f)). In particular, domains derived from the CD3ζ chain (ITAMs, feature e) and from the CD3ε chain (SEQ ID NO:1, feature f)). CD3ζ and CD3_{ε} chains are two independent peptides chains of the T cell receptor (TCR) complex. It is an essential feature of the present invention that the activation domain derived from the CD3ε chain comprises the amino acid sequence RKGQRDLY (SEQ ID NO:1) and might exclude the amino acid sequence NQRRI (endoplasmic reticulum retention signal) (SEQ ID NO:3).

The CAR according to the present invention has two activation domains preferably derived from the T cell receptor (TCR). Preferably one domain is derived from the CD3ζ chain, which is a TCR activatory domain (ITAM, feature e)) and a second domain is preferably derived from the CD3ε chain. This second domain (feature f)) contains a sequence that binds a tyrosine kinase in a phospho-tyrosine-independent manner via its SH3 domain. This domain (feature f)) which is preferably derived from the CD3ε chain comprises the amino acid sequence RKGQRDLY (SEQ ID NO:1). Furthermore, in a preferred embodiment this sequence does not include an amino acid sequence coding for the endoplasmatic reticulum retention signal having the amino acid sequence NQRRI (SEQ ID NO:3).

The chimeric antigen receptor (CAR) is provided by inserting the genetic information coding for such chimeric antigen receptor into a suitable vector. Such vector may be a plasmid or a vector derived from a virus preferably a lentiviral vector. The CAR encoded by a lentiviral vector may thus be used for the transfection of T cells. In a preferred embodiment of the present invention, the T cells of a patient to be treated are isolated ex vivo by a suitable leukapheresis proceeding. Such T cells are in vitro transfected with the suitable vector, preferably the lentiviral vector, and the transfected cells are isolated and amplified in culture. After the amplification of the genetically modified T cells, such cells are applied to a patient wherein the cells can act and kill the undesired cells. The T cells bind to the target cells (e.g. tumor cells) and are killing them.

The CARs according to the present invention are therefore suitable for use in the treatment of diseases. Such diseases are in particular various forms of cancer. The CARs according to the present invention may also be used in the treatment of other diseases, which are difficult to treat like chronic infections or autoimmune disorders or the treatment of severe viral infections. Likewise, more complex platforms such as the “T cell receptor (TCR) fusion protein” or “TFP” Platform or “TRuC” could be used for similar pathological scenarios. A “TFP” as referred to herein includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. A “TFP T Cell,” as used herein, is defined as an immune cell comprising a T cell receptor complex wherein one or more TCR or CD3 subunits has been engineered to include a binding moiety, e.g., an antibody or fragment thereof. In some embodiments, the antibody or fragment thereof comprises an scFv. In other embodiments, the antibody or fragment thereof comprises a single domain antibody (sdAb), e.g., a camelid or fragment thereof. In some embodiments, the binding moiety targets tumor-cell specific antigens, as are described herein. Exemplary tumor-cell specific antigens specifically bound by the binding moiety of a TFP or a CAR include, but are not limited to, CD19, BCMA, MUC16, MSLN, CD20, CD22, CD70, CD79B, HER2, and PSMA. The engineered subunit comprises (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR alpha or TCR beta (or, alternately, TCR gamma or TCR delta), and optionally a sequence encoding a TCR constant domain, wherein the TCR constant domain is a TCR alpha constant domain, a TCR beta constant domain or a TCR alpha constant domain and a TCR beta constant domain; wherein the TCR subunit and the antigen domain are operatively linked, and wherein the TFP functionally incorporates into a TCR complex when expressed in a T cell. The TFP T cell platform is described in International Patent Publication Nos. WO2018098365,WO2016187349, WO2018067993, and WO2018026953. In some embodiments, the TCR extracellular domain is selected from a TCR alpha, TCR beta, TCR delta, TCR gamma, CD3 epsilon, CD3 gamma, or CD3 delta extracellular domain. In some embodiments, the transmembrane domain is selected from a TCR alpha, TCR beta, TCR delta, TCR gamma, CD3 epsilon, CD3 gamma, or CD3 delta transmembrane domain. In some embodiments, the intracellular domain comprises a stimulatory domain from an intracellular signaling domain of CD3 epsilon. In some embodiments, the intracellular domain comprises a stimulatory domain from an intracellular signaling domain of CD3 epsilon comprising a duplication of the CD3 epsilon RKGQRDLY (SEQ ID NO:1) sequence.

Such modified T cells having a CAR exhibit highly desirable properties. The antigen-recognition domain is preferably a single-chain variable fragment (scFv) which can be derived from a suitable monoclonal antibody and which may preferably target a tumor-associated antigen. This domain can be, for example derived from an antibody recognizing CD19 in the context of a treatment of B-cell malignances such as lymphoma. It can, however, also bind to other antigens which are specific for tumor cells like PSA or PSMA for prostate cancer. The term “antigen-recognition domain” comprises also modified antigen binding sequences, in particular when the sequence is derived from mouse monoclonal antibodies. For the application to humans such sequences are preferably humanized in order to avoid unspecific immune reactions against sequences, which are recognized by the human immune system as foreign (mouse).

Without wishing to be bound to a theory it is assumed that the mechanism on which the invention is based has to do with a non-canonical binding of the LCK SH3 domain to a sequence contained in CD3ε (SEQ ID NO:1) which redefines initiation of TCR signaling. The decisive step of the invention is introducing a sequence in the cytoplasmic tail of the CAR or TFP that binds a tyrosine kinase via its SH3 domain and thereby, mediates proximity between the ITAMs of the receptor (CAR or TFP) and a tyrosine kinase, and thereby facilitates its phosphorylation. While the disclosure herein is exemplified primarily by the RKGQRDLY (SEQ ID NO:1) sequence in CD3ε, other phosphor-tyrosine independent and proline-dependent or proline-independent sequences such as, but not limited to sequences of the CD28 coreceptor, the UNC119 adaptor protein, the immune adaptor SKAP55, TRABID, Flavocytochrome c fumarate reductase, nuclear FMPR interacting protein I or the viral proteins, Tip and Nef, or a combination thereof may also be used.

The functionality of the amino acid sequence RKGQRDLY (SEQ ID NO:1) is defined by the minimal motif RK/Rx₁x₂R/Q/Yx₃x₄Y, with “x₁” being preferentially G, but in some cases could also be a positively charged amino acid (K, R or H); “x₂” being preferentially Q, but in some cases could also be any amino acid that is not positively charged (D, E, N, M, L, I, V, A, C, P, G, S, T, F, Y, W); “x₃” being preferentially a negatively charged amino acid (D or E), but in some cases could also be any uncharged amino acid (N, M, L, I, V, A, C, P, G, S, T, F, Y, W) and “x₄” being any uncharged amino acid (N, M, L, I, V, A, C, P, G, S, T, F, Y, W). Therefore, also slight modifications of SEQ ID NO:1 might show a similar effect. The changes should, however, be only minor whereby preferably not more than one amino acid of SEQ ID NO:1 is replaced.

Several experiments have been performed to elucidate the mechanism of TCR signal initiation in T cells. The results of such experiments are summarized below and in particular in FIGS. 22-28.

### Functional Considerations:

The T cell antigen receptor (TCR) is a multimeric protein complex expressed on the surface of T cells. The αβ TCR is composed of the ligand-binding TCRαβ heterodimer and the signal-transducing CD3 complex. In contrast to TCRαβ, CD3 subunits contain immunoreceptor tyrosine-based activation motifs (ITAMs) in their cytoplasmic tails that mediate signal transduction. Upon TCR binding to its ligand (major histocompatibility complex bound to an antigenic peptide, MHCp), the ITAMs are phosphorylated on tyrosine residues by the sarcoma (SRC) family kinase, LCK, and TCR signaling is initiated. In the absence of this phosphorylation, T cells do not get activated.

Lymphocyte specific Src kinase (LCK) is a modular protein, which plays an important role in the present invention. An acylated N-terminal part attaches LCK to the plasma membrane. A unique domain mediates LCK binding to the CD4 and CD8 coreceptors. An SH3 domain mediates binding to proline-rich sequences, whose basic sequence is PxxP, and an SH2 domain mediates phosphotyrosine interactions. LCK harbors a kinase domain containing the activating tyrosine, Y394, and a short C-terminal regulatory tail containing the inhibitory tyrosine, Y505.

LCK catalytic activity is inhibited by cooperative, intramolecular interactions between the SH3 domain and a proline-rich sequence situated in the linker between the SH2 and kinase domains, and between the SH2 domain and the phosphorylated Y505. This autoinhibited kinase is a precariously set mousetrap, in which alternative interactions can be sufficient for activation. SH3 domain displacement via binding to PxxP sequences in the CD28 coreceptor, the UNC119 adaptor protein or the viral proteins, Tip and Nef, activates LCK. Phosphorylation of Y394 is the strongest activator of LCK catalytic activity.

A mechanistic explanation of how LCK is recruited to the ligated TCR to initiate CD3 phosphorylation remains elusive. Among the existing models, it has been proposed that the formation of a trimolecular complex of the TCR with MHCp and the CD4 or CD8 coreceptors brings coreceptor-associated LCK molecules close to the CD3 cytoplasmic tails to initiate phosphorylation. However, recent reports show that complex formation occurs in two stages. The MHCp ligand first binds the TCR and results in CD3 ITAM phosphorylation by a free, rather than coreceptor-bound, pool of LCK. The subsequent MHCp-coreceptor binding recruits the coreceptor-associated LCK via interaction between the LCK SH2 domain and the phosphorylated ITAMs, and increases T cell sensitivity. That the proportion of Y394-phosphorylated LCK remains constant upon ligand binding to the TCR supports an alternative triggering hypothesis, which proposes that spatial reorganization of LCK within the plasma membrane might play an important role in TCR signaling. Other mechanisms for TCR triggering have also been proposed. Segregation of phosphatases from the vicinity of ligated TCRs might favor accumulation of phosphorylated ITAMs. TCR phosphorylation might also be controlled by conformational changes within the receptor regulating the accessibility of the CD3 ITAMs to LCK. While these models theorize mechanistic steps for ligand triggering of TCR signaling, they say nothing about how LCK is targeted to the TCR to initiate CD3 phosphorylation.

T cell-based immunotherapy using chimeric antigen receptors (CARs) is a major breakthrough in the treatment of cancer providing hope for curative responses in patients with hematological malignances. CARs are synthetic receptors redirecting T cells to mediate tumor rejection by combining signaling domains from the TCR (namely the cytoplasmic tail of the ζ chain) and from co-stimulatory receptors (such as 41BB). ITAM phosphorylation is also crucial for signal initiation of CARs. How LCK phosphorylates the ITAMs of these chimeric receptors remains unknown.

Without wishing to be bound to a theory, the chimeric receptor according to the present invention rationally integrates the domain that recruits LCK to phosphorylate the ITAMs to a chimeric receptor to improve anti-tumor functionality of chimeric antigen receptor (CAR).

### Experimental Results are Mainly Shown in the Figures:

According to the present invention, the RK motif

(sequence RKGQRDLY, SEQ ID NO: 1)

was introduced in a well-characterized anti-CD19 CAR (from now on called ζCAR, FIG. 1). The starting construct was described by Imai et al., Leukemia, 2004, 18, 676-684, and Maude et al., N. Engl. J. Med. 2018, 378, 439-448. We aim, to analyze the impact of LCK recruitment to chimeric receptors on the anti-tumor functionality of T cells. This approach serves as proof-of-principle of the applicability of adding a complementary TCR-derived activatory domain include in preferred embodiments the amino acid sequence RKGQRDLY (SEQ ID NO:1).

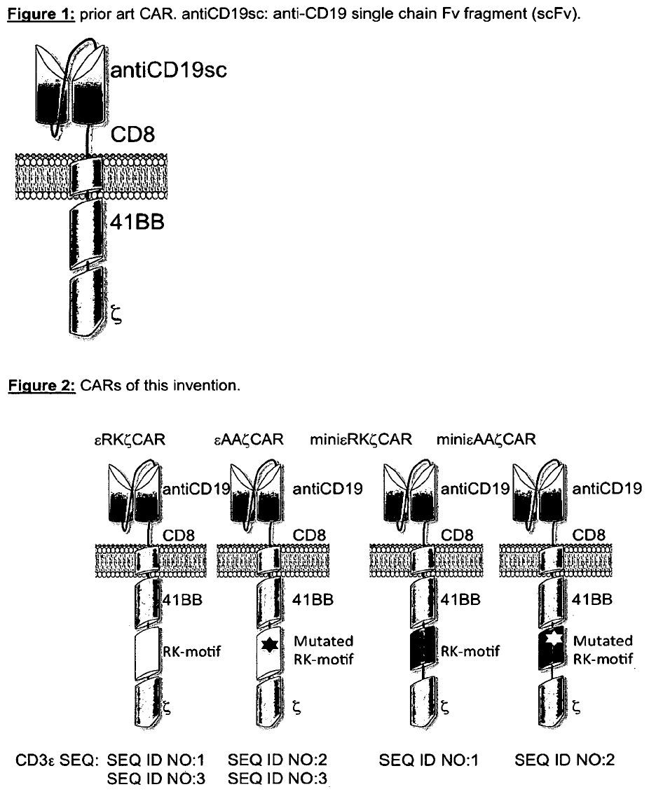

Four chimeric antigen receptors were designed (FIG. 2), which are based on the well-characterized anti-CD19 CAR (here named called ζCAR, FIG. 1, (Imai et al., 2004; Maude et al., 2018)).:

1. By adding part of the CD3ε cytoplasmic tail including the RK motif

(sequence RKGQRDLY, SEQ ID NO: 1)

between the 41BB and ζ sequences. This chimeric receptor will be named εRKζCAR.

2. By adding part of the CD3ε cytoplasmic tail including a mutated RK motif

(sequence AAGQRDLY, SEQ ID NO: 2)

between the 41BB and ζ sequences. This chimeric receptor will be named εAAζCAR.

3. By adding part of the CD3ε cytoplasmic tail including the RK motif

(sequence RKGQRDLY, SEQ ID NO: 1)

excluding the endoplasmatic reticulum retention signal having the amino acid sequence NQRRI (SEQ ID NO:3). This chimeric receptor will be named miniεRKζCAR.

4. By adding part of the CD3ε cytoplasmic tail including a mutated RK motif

(sequence AAGQRDLY, SEQ ID NO: 2)

between the 41BB and sequences and ζ sequences and excluding the endoplasmatic reticulum retention signal having the amino acid sequence NQRRI (SEQ ID NO:3). This chimeric receptor will be named miniεAAζCAR.

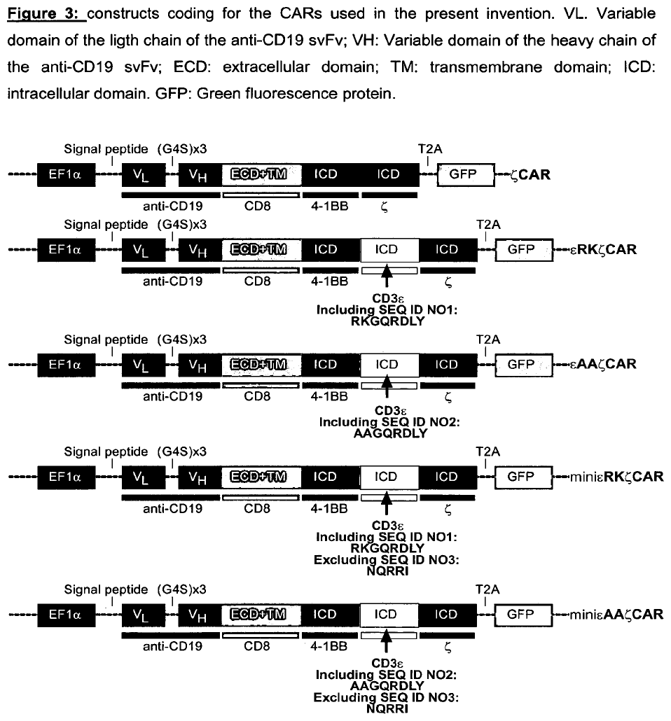

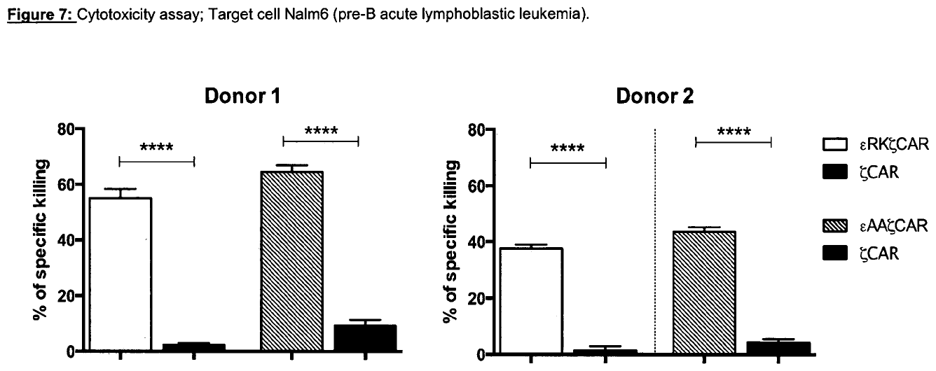

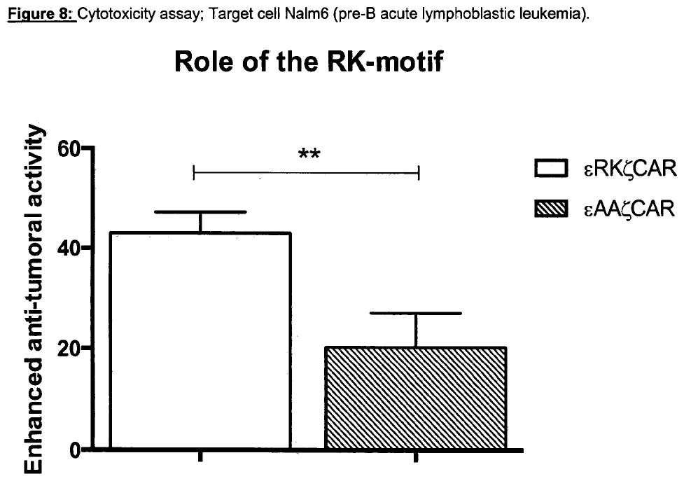

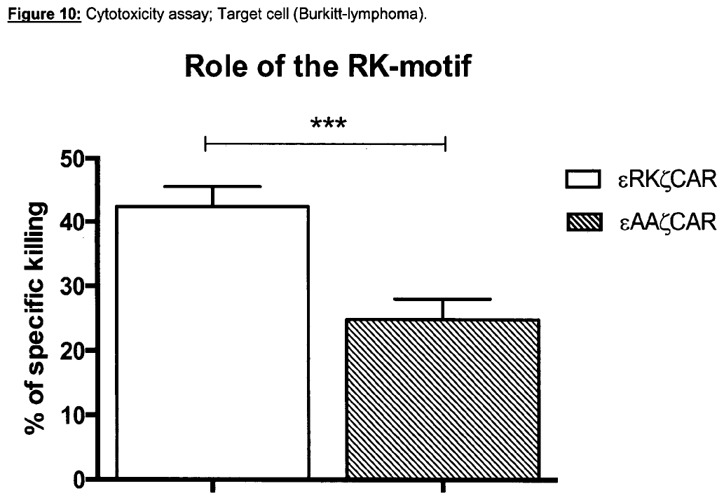

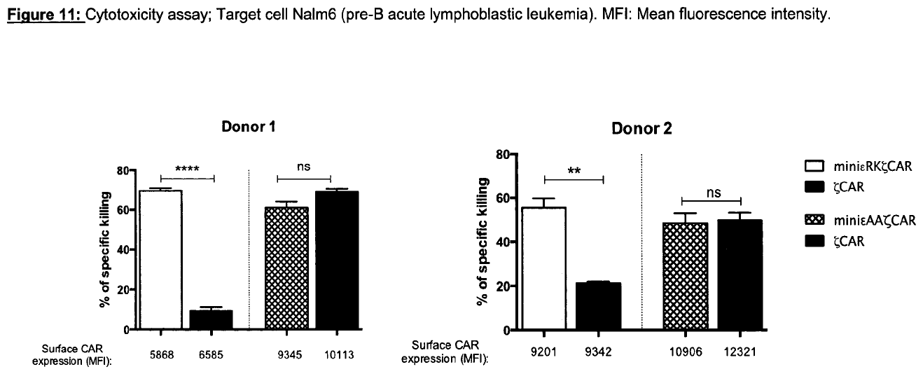

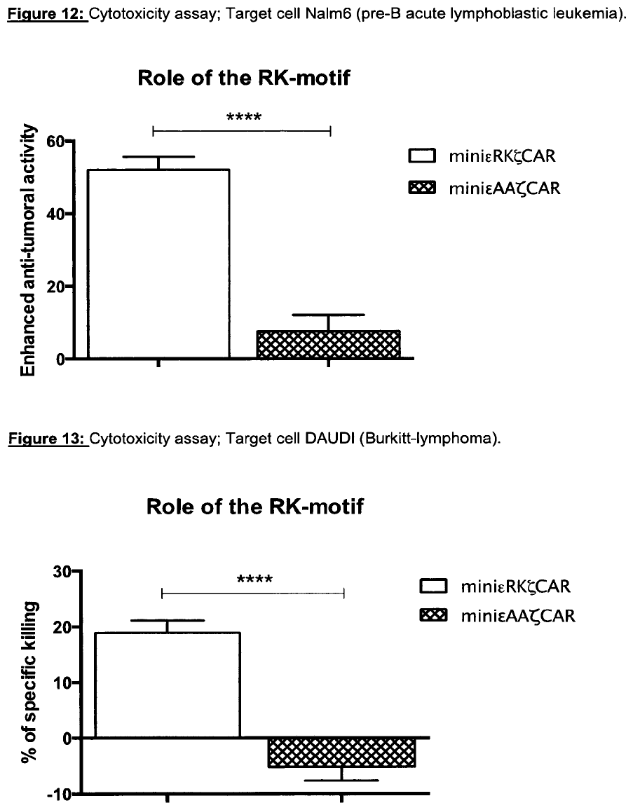

These four chimeric antigen receptors were used in the present invention and compared to the well-characterized anti-CD19 CAR (here named called ζCAR, FIG. 1, (Imai et al., 2004; Maude et al., 2018)). The results are described in more detail in the following FIGS. 1-17).

In the course of the present invention several experiments have been performed and the results are shown in the FIGS. 1-28. The details of the experiments and the Figures relate to the present invention unless expressly stated. Important aspects of the present invention are summarized in the following Tables:

TABLE 1
The sequences used in this invention

| SEQ ID NO: | Amino acid sequence | Origin/Comments |
|---|---|---|
| 1 | RKGQRDLY | Human CD3ϵ/here named RK motif |
| 2 | AAGQRDLY | Mutation of SEQ ID NO: 1 |
| 3 | NQRRI | Human CD3ϵ (ER retention signal) |

TABLE 2
Correlation between Figures and experiments

| | | Related to |
|---|---|---|
| Figure: | Title/Description | Experiment |
| 1 | prior art CAR | Experiment 1-6 |
| 2 | CARs of this invention (with controls) | Experiment 1-6 |
| 3 | Constructs coding for the CARs used | Experiment 1-6 |
| | in the present invention | |
| 4 | Level of CAR surface expression in | Experiment 1 |
| | CAR T cells | |
| 5 | Cytotoxicity assay; CARs; Target | Experiment 2 |
| | cell Nalm6 | |
| 6 | Cytotoxicity assay; CARs; Target | Experiment 3 |
| | cell DAUDI | |
| 7 | Cytotoxicity assay; CARs; Target | Experiment 2 |
| | cell Nalm6 (Alternative analysis) | |
| 8 | Cytotoxicity assay; CARs; Target | Experiment 2 |
| | cell Nalm6 (Alternative analysis) | |
| 9 | Cytotoxicity assay; CARs; Target | Experiment 3 |
| | cell DAUDI (Alternative analysis) | |
| 10 | Cytotoxicity assay; CARs; Target | Experiment 3 |
| | cell DAUDI (Alternative analysis) | |
| 11 | Cytotoxicity assay; CARs; Target | Experiment 2 |
| | cell Nalm6 (Alternative analysis) | |
| 12 | Cytotoxicity assay; CARs; Target | Experiment 2 |
| | cell Nalm6 (Alternative analysis) | |
| 13 | Cytotoxicity assay; CARs; Target | Experiment 3 |
| | cell DAUDI (Alternative analysis) | |
| 14 | Exhaustion markers in the absence | Experiment 4 |
| | of target cells; CARs | |
| 15 | Time line of the pre-clinical model; | Experiments 5 |
| | CARs | and 6 |
| 16 | Stress-test in vivo; CARs | Experiment 5 |
| 17 | Pre-clinical model in vivo; CARs | Experiment 6 |
| 18 | prior art TRuC | Experiment 7 |
| | | and 8 |
| 19 | TRuC of this invention (2xRKTRuC) | Experiment 7 |
| | | and 8 |
| 20 | TRuC surface expression in | Experiment 7 |
| | primary T cells | |
| 21 | Cytotoxicity assay; TRuCs; Target | Experiment 8 |
| | cell Nalm6 | |

In the course of the present invention, it was investigated whether LCK localization in respect to the TCR changes upon ligand binding to the TCR. The proximity of the CD3δ cytoplasmic tail to LCK was analyzed in intact cells. The in situ proximity ligation assay was used, which lacks the drawbacks of detergent lysis. The presence of a fluorescent dot indicates that the distance between one CD35δ and one LCK protein is less than 80 nm. Several controls demonstrated the specificity of this approach. In resting T cells, TCR:LCK interactions were above background readings in the assay, indicating that a detectable proportion of LCK molecules are near the TCR. To investigate whether this proximity changes upon TCR stimulation, the assay was performed before and after TCR stimulation at 37° C. Anti-human CD3ε (anti-CD3ε, OKT3) antibody stimulation was used to solely focus on the proximity between LCK and TCR without involving other LCK-interacting proteins such as CD4, CD8 or CD28. The number of LCK molecules in close proximity to TCR molecules increased upon stimulation in primary human T cells. Similar results were obtained in concert with pervanadate treatment, which inhibits phosphatases inducing TCR phosphorylation. Stimulation with anti-CD3 antibodies at 37° C. results in ITAMs phosphorylation, meaning that the LCK SH2 domain could have bound to the phosphorylated ITAMs to achieve the proximity shift.

The TCR lacks intrinsic kinase activity, and cannot phosphorylate itself to generate the first docking site for the LCK SH2 domain. It is assumed that LCK recruitment to the TCR must, therefore, precede any phosphorylation event. To mimic phosphorylation-independent conditions during analysis of CD3δ: LCK proximity, analyses were performed in the presence of the SRC kinase inhibitor, PP2. Strikingly, the number of TCR:LCK interactions significantly increased upon stimulation with anti-CD3ε in primary human T cells. Antibody binding mimics antigen binding and results in a reversible conformational change in the CD3 cytoplasmic tails (CD3CC) that stabilizes the active TCR conformation prior to ITAM phopsphorylation. Treatment with pervanadate at 4° C. in the presence of PP2 served as a control in which neither the active TCR conformation is stabilized nor ITAM phosphorylation takes place. The extent of basal TCR:LCK interactions upon pervanadate treatment was similar to unstimulated samples, demonstrating that the tests were indeed performed under SRC kinase phosphorylation-free conditions. The data demonstrate that LCK is recruited to active TCRs before ITAM phosphorylation occurs (FIG. 22).

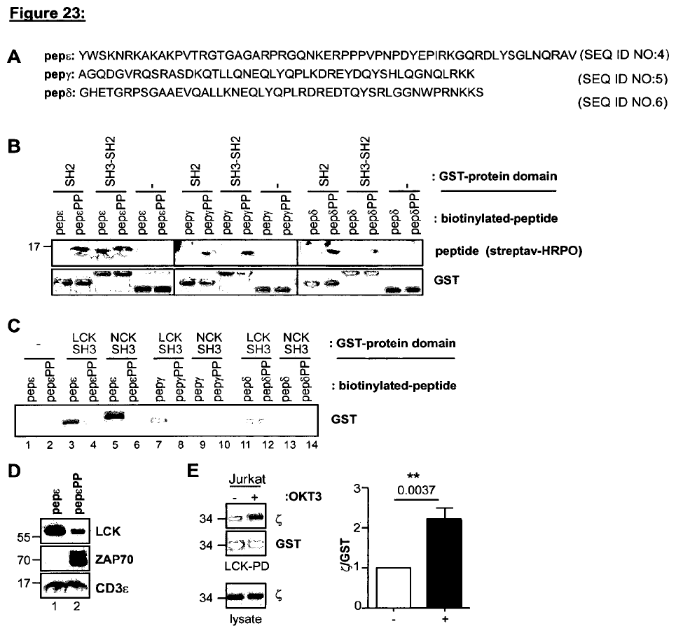

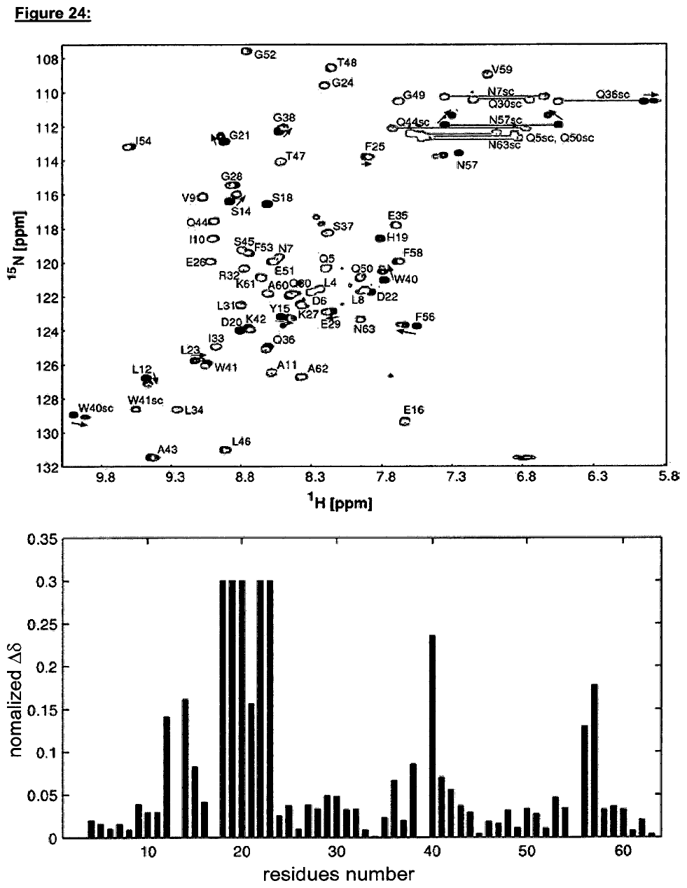

To identify the LCK protein domain responsible for binding to the non-phosphorylated TCR in the active TCR conformation, associations were tested between either LCK SH2 or SH3 domains and peptides containing the complete CD3 cytoplasmic tails either with or without ITAM phosphorylation. Consistent with the pervanadate-derived results, the LCK SH2 bound to the phosphorylated CD3ε, CD3γand CD3 δ peptides. A fusion protein containing both the LCK SH3 and SH2 domains also bound to non-phosphorylated CD3ε, but not to non-phosphorylated CD3γ and CD3δpeptides. SH2 domains bind to phosphorylated tyrosines, and therefore, they cannot mediate the observed binding to non-phosphorylated CD3ε. Hence, it was tested whether the LCK SH3 domain alone can bind to the CD3 cytoplasmic tail. The NCK SH3.1 domain was used, which binds to the CD3E proline-rich sequence, as a positive control. Indeed, the LCK SH3 domain bound to the non phosphorylated CD3ε cytoplasmic tail, suggesting that this interaction mediates LCK recruitment to the TCR. Incubation of Jurkat T cell lysates with phosphorylated and non-phosphorylated CD3ε peptides showed that endogenous full length LCK bound both phosphorylated and non-phosphorylated CD3ε peptides. As expected, ZAP70 bound only the phosphorylated CD3ε peptide. Lysates from unstimulated Jurkat T cells or Jurkat T cells stimulated with anti-CD3ε were next incubated with LCK SH3 domain-coated beads. Antibody stimulation resulted in increased TCR binding to LCK SH3. Altogether, these data indicate that the LCK SH3 domain binds to the non-phosphorylated CD3ε cytoplasmic tail upon TCR stimulation (FIG. 23).

To gain insights into the molecular details of the LCK SH3 interaction with CD3ε, nuclear magnetic resonance (NMR) spectroscopy was used. The LCK SH3 domain and the CD3ε cytoplasmic tail were expressed in E. coli. Spectral changes of 15N-labeled LCK SH3 were observed in a series of 1H, 15N heteronuclear single-quantum correlation experiments (HSQC) with addition of the unlabeled CD3ε cytoplasmic tail in molar excess. Prominent signal changes were observed for the S18-L23, W40 and F56-N57 residues in the LCK SH3. These chemical shift changes conclusively demonstrate an interaction between LCK SH3 and CD3ε (FIG. 24).

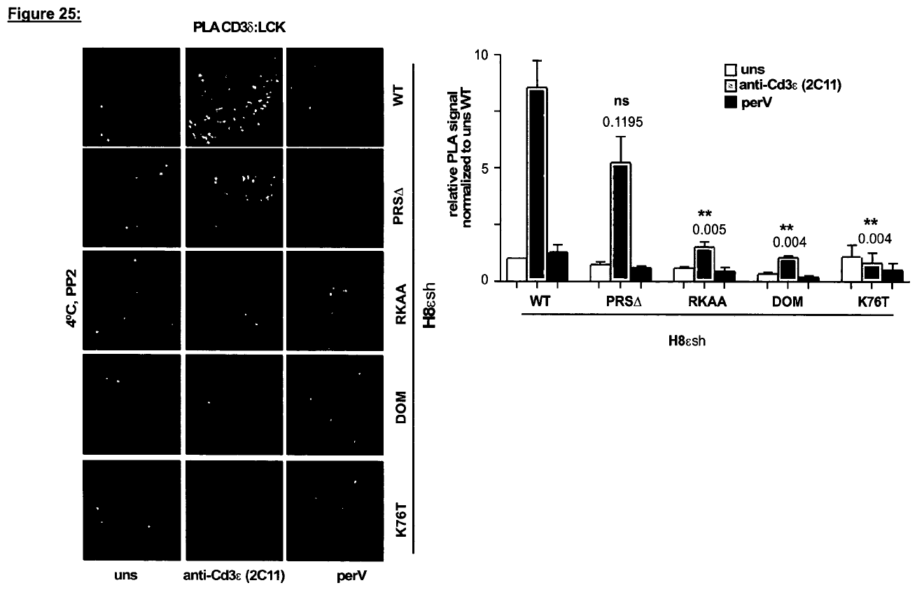

SH3 domains bind to proline-rich sequences, and CD3ε contains the proline-rich sequence (PRS), PxxPxxDY, which is only accessible in the active TCR conformation. To investigate whether LCK binds PxxPxxDY, Jurkat-derived, CD3ε-silenced cell lines (named H8εsh cells) were generated expressing murine wildtype (WT) CD3ε, mutant Cd3ε containing the proline-depleted AxxAxxDY motif (PRSA), which was described to abolish SH3 NCK binding, or Cd3ε with the K76T mutation, which prevents the TCR from switching to its active conformation. It was first confirmed that WT and mutant Cd3ε molecules are expressed and behave as expected once expressed in the cells (such data are not shown in a Figure). These new cell lines allowed to test whether the CD3ε PxxPxxDY binds to LCK in the absence of ITAM phosphorylation. Only the introduced murine Cd3 molecules were stimulated by incubating the cells with an anti-Cd3ε antibody (145-2C11) at 4° C. in the presence of PP2, and the CD3δ: LCK proximity ligation assay was performed. Unexpectedly, TCR:LCK interactions increased similarly in both H8εsh cells expressing Cd3εPRSΔ or Cd3εWT upon antibody-mediated stabilization of the active TCR conformation. Contrastingly, hardly any proximity was detected between the TCR and LCK in H8εsh cells expressing Cd3εK76T. Taken together, these data demonstrate that the TCR in its active conformation exposes a yet unidentified LCK binding site in CD3ε (FIG. 25).

Although most SH3 domains bind the core PxxP motif, some exceptions have been reported. The FYB SH3 domain binds the proline independent RKxx(Y)xxY motif in the SKAP1 adaptor protein, to which the SH3 domains of FYN and LCK bind with 10-fold less affinity. The related

(SEQ ID NO: 1)
RKGQRDLY

sequence was identified, which is conserved from lobe-finned fishes to mammals, in the CD3ε cytoplasmic tail. To investigate whether this sequence might interact with the LCK SH3 domain, RK was mutated to AA while leaving the rest of the sequence intact:

(SEQ ID NO: 2)
AAGQRDLY.

The mutated Cd3ε (Cd3εERKAA) was expressed in H8εsh cells, and performed the proximity ligation assay under phosphorylation-free conditions. A double mutant combining RKAA and PRSΔ mutations was also created (Cd3εDOM). All cell lines were characterized. In contrast to the cells expressing WT Cd3εor Cd3εPRSΔ, hardly any proximity between the TCR and LCK was detected in H8εsh cells expressing Cd3εRKAA or Cd3εDOM. Taken together, these data indicate that the ligated TCR exposes the RK sequence, which mediates LCK SH3 domain binding and, thereby, LCK recruitment to the TCR prior to any ITAM phosphorylation (FIG. 25).

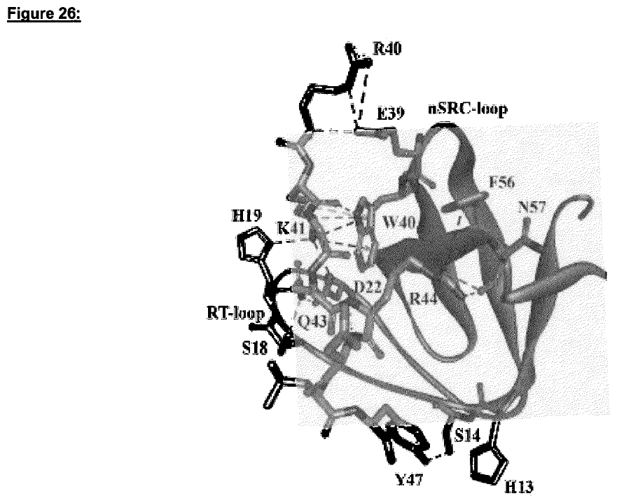

To gain molecular insights into this non-canonical interaction, a protein-protein docking simulation was performed with the CD3ε cytoplasmic tail and the LCK SH3 domain as input structures using the HADDOCK docking web server. The RKGQRDLY (SEQ ID NO:1) sequence in CD3ε and L12, S14, S18, H19, D20, G21, D22, L23, W40, F56, and N57 residues in the LCK SH3 domain were used as ambiguous interaction restraints. The top-ranked model for docking showed that the CD3ε R40, K41, Q43, R44, and Y47 residues interacted with H13, S14, S18, H19, D22, E39, W40, F56, and N57 in LCK SH3. R40 interacted with E39 via a hydrogen bond and an electrostatic interaction. K41 mediated a salt-bridge interaction with D22, hydrogen bond interactions with H19, D22, and W40, and a network of hydrophobic and electrostatics interactions with W40. These anchorages of the LCK SH3 domain in the RT and n-SRC loops support a critical role of the CD3ε R40 and K41 residues in LCK SH3 binding to CD3E. Q43 interacts with S18 and D22 via hydrogen bonds, R44 interacts with F56 via a π-cation interaction and with N57 via a hydrogen bond and Y47 forms a hydrogen bond with S41. This Y47-S41 interaction using the CD3ε Y47 hydroxyl group is abolished upon Y47 phosphorylation, explaining why the LCK SH3 domain failed to bind the phosphorylated CD3ε peptide. We speculate that Y47 phosphorylation might mechanistically explain the transient interaction of LCK to the ligand-bound TCR. Altogether, these results show that the RKGQRDLY (SEQ ID NO:1) sequence (now called the Receptor Kinase interaction or RK motif) in CD3ε binds to the LCK SH3 domain (FIG. 26).

To investigate whether LCK binding to the

(SEQ ID NO: 1)
RKGQRDLY

sequence in CD3ε increases LCK activity at the TCR, a reporter for LCK activity was designed that is only phosphorylated if active LCK is in the vicinity of the TCR. This reporter consists of an inactive LCK kinase domain (K273A mutant) coupled to the CD3ε chain by a flexible linker, and fulfills the following criteria:
(i) the K273A mutation keeps the LCK kinase domain enzymatically inactive,
(ii) Y394 in the LCK kinase domain is a well characterized substrate for LCK, whose phosphorylated form can be detected with specific antibodies and
(iii) the LCK kinase domain is constitutively exposed.

This reporter (referred to as ζ-Reporter) was expressed in ζ-deficient MA5.8 murine T cells, and cells were analyzed for Cd3 ITAM and reporter phosphorylation. In unstimulated T cells, phosphorylation of the ζ-Reporter at the kinase-dead domain (pY394) was readily detectable, suggesting that active LCK is in close proximity to the TCR in resting T cells.

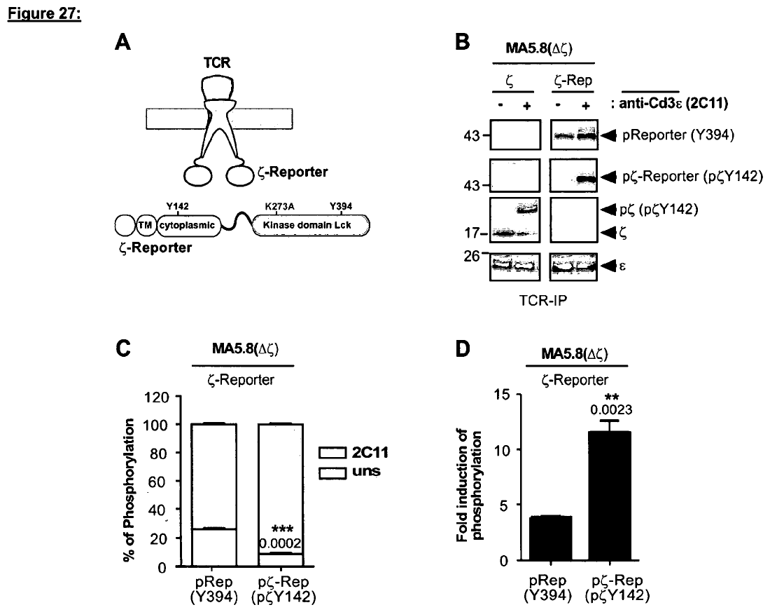

Despite that, hardly any phosphorylation at the ζ ITAMs (pζY142) was observed, indicating that in the resting TCR, the ITAMs are protected from phosphorylation. Phosphorylation of Y394 in the ζ-Reporter, was inhibited by PP2, supporting that LCK was the phosphorylating kinase (data not shown). On the one hand, TCR stimulation resulted in a 4-fold increase of Y394 phosphorylation of the ζ-Reporter despite being constantly accessible. These data demonstrate that LCK activity at the TCR increased upon TCR activation, most probably by LCK recruitment to the RK motif in the ligated TCRs. On the other hand, TCR stimulation resulted in a larger increase in ITAM phosphorylation, namely 11-fold. These data suggest that a pool of active LCK is close to the resting TCR, in which the ITAMs are protected from spurious phosphorylation. In conclusion, antibody-mediated stabilization of the active TCR conformation leads to a local increase of LCK activity at the TCR and to the exposure of the ITAMs for phosphorylation (FIG. 27).

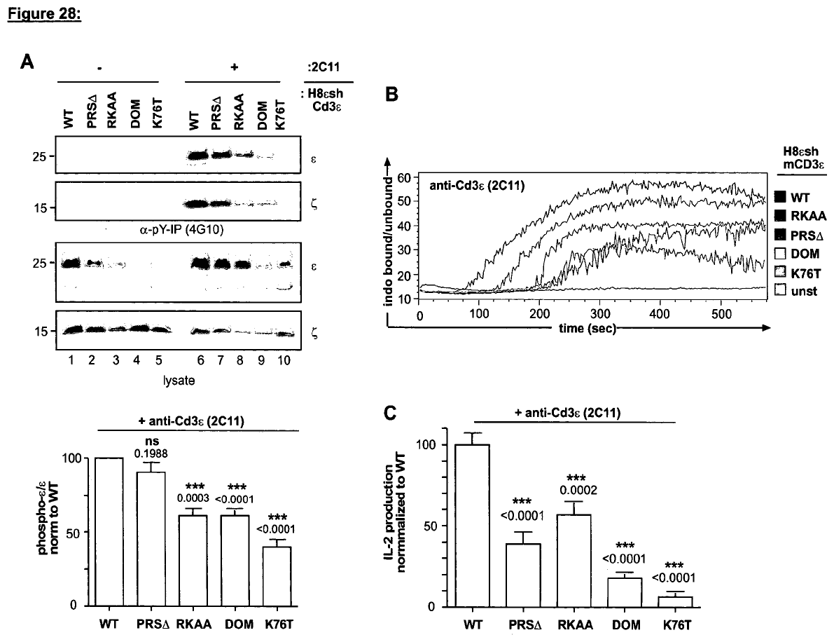

To test a functional implication of the LCK SH3 domain recruitment to the RK motif in T cell activation, the CD3δ: LCK proximity ligation assay was performed under more physiological conditions, namely at 37° C. in the absence of kinase inhibition. In Cd3εWT-or Cd3εPRSΔ-expressing cells, the basal TCR:LCK proximity increased after stimulating both with antibodies or pervanadate. In contrast, hardly any TCR:LCK interaction was observed in cells expressing the Cd3RKAA mutant or the CD3CC-deficient TCR. Next, the role of the RK motif in CD3 phosphorylation was investigated. Cells reconstituted with Cd3εWT showed strong TCR phosphorylation upon stimulation. In contrast, TCR phosphorylation was reduced by 40% in Cd3εRKAA-and in Cd3εDOM-expressing cells compared to Cd3εWT cells. TCR phosphorylation was decreased by 60% in CD3CC-deficient cells. These data indicate that the RK motif is required to initiate optimal TCR phosphorylation. The influence of the RK motif on downstream TCR signaling was also tested, such as calcium influx and IL2 production. Mutation of the RK motif resulted in a delayed and decreased calcium response upon antibody stimulation. Cells expressing Cd3εPRSΔ exhibited reduced TCR-induced calcium influx, most likely because the interaction of the TCR with NCK was prevented. Expression of Cd3εDOM and Cd3εK76T caused a further reduction and delay of calcium influx. Finally, mutation of the RK or proline-rich sequence motifs caused reduced IL2 production upon stimulation with anti-Cd3ε and anti-Cd28 antibodies. This reduction was enhanced in the Cd3εDOM and in CD3εCC-deficient cells. Taken together, these data indicate that the RK motif plays an important role in stimulation-induced phosphorylation of the CD3 cytoplasmic tails impacting downstream TCR signaling (FIG. 28).

An important aspect of the present invention is that the RK motif (SEQ ID NO:1) is essential for efficient signaling in antigen-induced TCR activation.

In the present invention is shown how LCK is targeted to the TCR, and this is used in a new concept of signal initiation. LCK is recruited to the ligated TCR by direct binding of the LCK SH3 domain to the CD3ε cytoplasmic tail prior to receptor phosphorylation. SH3 domains commonly bind to PxxP sequences, and CD3ε proline-rich sequence exposure is the best-characterized hallmark of the ligand-bound TCR. LCK recruitment occurred despite mutation of the proline-rich sequence in the experiments. NMR spectroscopy and molecular modelling demonstrated that the non-canonical SH3-binding motif, the

(SEQ ID NO: 1)
RKGQRDLY

sequence, in the CD3ε cytoplasmic tail, which is termed the RK motif, binds to both the RT-loop and n-SRC loop of the LCK SH3 domain. Most LCK RT-loop residues are not conserved in FYN or other SRC family kinases, possibly explaining why FYN cannot substitute for LCK during T cell development and activation. Two related RK motifs have been identified, one in the SKAP1 adaptor protein (RKxx(Y)xxY), and another one in C. albicans. These are the first proline-independent SH3 binding motifs described. Their distribution across the evolutionary panorama suggests that SH3 domains may bind to non-canonical motifs more often than previously thought, redefining the one-key-for-one-lock view of protein-protein interaction.

RK motif mutation

(sequence AAGQRDLY, SEQ ID NO: 2)

prevented LCK binding to the TCR and reduced antibody-induced CD3 phosphorylation, calcium mobilization, and IL2 production involved with downstream signaling (FIG. 24). Similarly, overexpressing a peptide containing the related SKAP1 RK motif in murine splenocytes and human T cell lines attenuated II2 and IL2 transcription, respectively. However, TCR signaling was only completely blocked in cells expressing CD3CC-deficient or doubly mutated receptors, suggesting that CD3CC-mediated exposure of both the proline-rich sequence and RK motif is needed for CD3 phosphorylation. NCK1 recruitment to the proline-rich sequence has been shown to be important for TCR phosphorylation and downstream signaling, and LCK recruitment was hindered in cells expressing a mutated RK motif. Taken together, these data provide a mechanistic explanation to the complete block of receptor signaling observed in CD3CC-deficient TCRs, since these mutant receptors expose neither the proline-rich sequence nor the RK motif and recruit neither NCK1 nor LCK.

A proportion of LCK is active in resting T cells. It is assumed that LCK is close to the TCR in resting cells, whether this LCK is actually active, was unclear. For this reason, a novel reporter for LCK activity was developed to demonstrate that active LCK is in close proximity to the TCR in resting cells. This local LCK activity further increased 4-fold upon receptor stimulation. The individual contributions of LCK recruitment to the TCR (as detected by proximity ligation assay), of conformational changes of the recruited LCK molecules and of exclusion of phosphatases to the local enhancement of LCK activity should be investigated further. Conversely, phosphorylation of the CD3 ITAMs increased 11-fold, suggesting that other mechanisms beyond the increase in local LCK activity foster ITAM phosphorylation. These mechanisms might be TCR intrinsic, such as regulation of ITAM accessibility either by stabilization of the active TCR conformation or release of the CD3 cytoplasmic tails from the plasma membrane.

Altogether, the work forming the basis of the present invention summarized how phosphorylation of the TCR is initiated:
(i) ligand binding stabilizes the T cell receptor (TCR) in its active conformation in which the RK motif

(sequence RKGQRDLY, SEQ ID NO: 1)

and the CD3 ITAMs become accessible;
(ii) the LCK SH3 domain binds the RK motif, recruiting LCK to the TCR, and
(iii) recruited LCK phosphorylates the ITAMs.

The results clearly demonstrate that binding of LCK directly to the RK motif

(sequence RKGQRDLY, SEQ ID NO: 1)

triggers TCR signal initiation. Consequently this sequence is contained within the constructs of the invention.

The present invention is described in more detail in the experiments 1-8, whereby the results are already presented in figures.

In all experiments the following methods have been used:

### a) Generation of CAR Constructs

The lentiviral vector pCDH-EF1-19BBζ-T2A-copGFP (coding for the ζCAR) was obtained from TCR² therapeutics. Briefly, it is a second generation CAR consisting of the scFV from murine anti-CD19 (FMC63) fused to the extracellular and transmembrane part of human CD8 alpha (amino acids 138-206), the 41BB endodomain and the ζ cytoplasmic tail. To generate the εRKCAR and miniεRKζCAR constructs, a truncated cytoplasmic tail of human CD3ε (amino acids 153-207 and amino acids 153-202, respectively) was placed between 41BB and ζ. εAAζCAR and miniεAAζCAR constructs were generated by mutation of the εRKζCAR and miniεRKζCAR constructs, respectively, using a Site-Directed Mutagenesis kit (Agilent Technologie) according to the manufacturer's instructions. For the 2xRKTRuC, the lentiviral vector pCDH_EF1_epsilonTRuC (coding for the prior art TRuC) was used. It contains the scFV from murine anti-CD19 (FMC63) fused to the extracellular domain of human CD3e via a flexible linker (sequence: GGGGSGGGGSGGGGS). To generate the 2xRKTRuC a truncated cytoplasmic tail of human CD3ε (amino acids 190-202 (accession number: P07766), containing the SEQ ID NO:1) was placed in the cytoplasmic tail of CD3ε at the position after amino acid 202 (accession number: P07766).

### b) Lentivirus Production

10⁷ HEK293T cells were plated on a 15 cm plate in 20 ml DMEM medium, distributed evenly, and incubated at 37° C. and 7,5% CO₂. After 24 hours, medium was changed and HEK293T cells were transfected with the respective constructs and the packaging plasmids pMD2.G (envelope) and pCMVR8.74 (gag/pol) using PEI transfection.

The virus-containing supernatant was harvested 24 and 48 hours after transfection and was concentrated by a 10% sucrose gradient (supplemented with 0.5 mM EDTA) centrifugation for 4 hours at 10000 rpm and 8° C. After centrifugation, the supernatant was discarded and the virus pellet was resuspended in 100 μl of PBS and stored at −80° C.

### c) Primary Human T Cell Activation, Transduction, and Expansion

Peripheral blood mononuclear cell (PBMCs) were purified from fresh blood of healthy donors using density centrifugation (Ficoll-Paque). PBMCs were counted, resuspended in medium supplemented with 500 U/ml recombinant human IL2 (PeproTech) and activated with plate-bound anti-CD3/CD28 antibodies (1 μg/ml). 48-72 hours after activation, the remaining PBMCs are mostly T cells (>99%). Primary human T cells were then lentivirally transduced using spin infection in the presence of 5 μg/m1 protamine sulfate (Sigma), 1000 U/ml IL2 with a MOI of 5 (at least otherwise indicated). Transduced T cells were checked for CAR expression 5-7 days after transduction using a biotinylated primary goat anti- mouse F(ab′)² antibody (Invitrogen) followed by streptavidin-APC (Biolegend). Cells were cultured in medium supplemented with 100 U/ml IL2 for a maximum of 7 days after transduction before used for killing assays.

### d) Cytotoxicity Assay

Luciferase-expressing tumor cells (Nalm6 or DAUDI as indicated) were plated at a concentration of 1×10⁵ cells/ml in 96-well flat bottom plates in triplicates. 75 μg/ml D-firefly luciferin potassium salt (Biosynth) was added to the tumor cells and Bioluminescence (BLI) was measured in the luminometer (Tecan infinity M200 Pro) to establish the BLI baseline. Right after, CAR-T cells were added at 5:1 effector-to-target (E:T) ratio and incubated for 8 or 24 hours (as indicated) at 37° C. BLI was measured as relative light units (RLU). RLU signals from cells treated with 1% Triton X-100 indicates maximal cell death. RLU signals from tumor cells without CAR-T cells determine spontaneous cell death. Percent specific

lysis was calculated with the following formula: % specific lysis=100×(average spontaneous death RLU−test RLU)/(average spontaneous death RLU−average maximal death RLU).

By using the above shortly described general methods the following experiments were performed:

### Experiment 1:

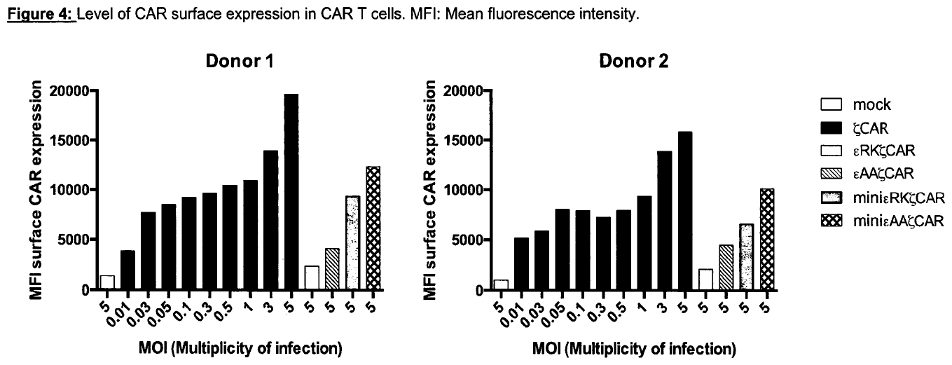

Expanded human T cells from healthy donors were transduced with a lentiviral vector coding for the ζCAR at different multiplicities of infection (MOI) to obtain different levels of expression of the ζCAR on the cell surface. Increasing the MOI elevated the level of ζCAR expression as tested by flow cytometry using fluorescent-labelled antibodies against the extracellular part of ζCAR. Expanded human T cells from healthy donors were also transduced with lentiviral vectors coding for the embodiments of the present invention εRKζCAR, εAAζCAR, miniεRKζCAR and miniεAAζCAR at MOI 5. The results are shown in FIG. 4. By choosing the appropriate MOls for the SζCAR, the prior art SζCAR can be compared with the constructs of the invention, which were expressed to a lower level.

### Experiment 2:

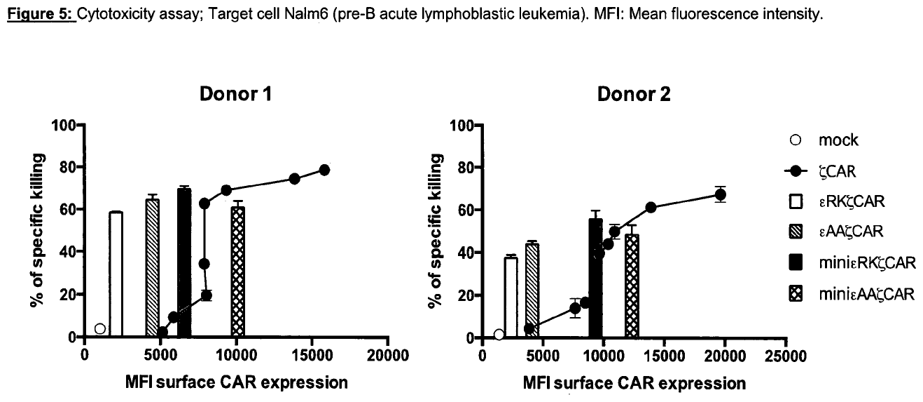

The anti-tumor activity (killing activity) of human T cells transduced with the lentiviral vector coding for the ζCAR at different multiplicities of infection (MOI) correlates with the level of surface expression of SCAR. Donors were healthy volunteers. The target cells were Nalm6 pre-B acute lymphoblastic leukemia. Expanded human T cells transduced with the lentiviral vector were incubated with the target cells at 5:1 effector-to-target (E:T) ratio and incubated for 8 at 37° C. The specific anti-tumor activity was calculated as indicated in the methods section (Cytotoxicity assay). Comparison of the CARs according to the invention to the prior art CAR (ζCAR) at the same level of surface expression demonstrates that the embodiments of the present invention εRKζCAR, εAAζCAR and miniεRKζCAR have a higher anti-tumoral activity. The results are shown in FIGS. 5, 7, 8, 11 and 12.

### Experiment 3:

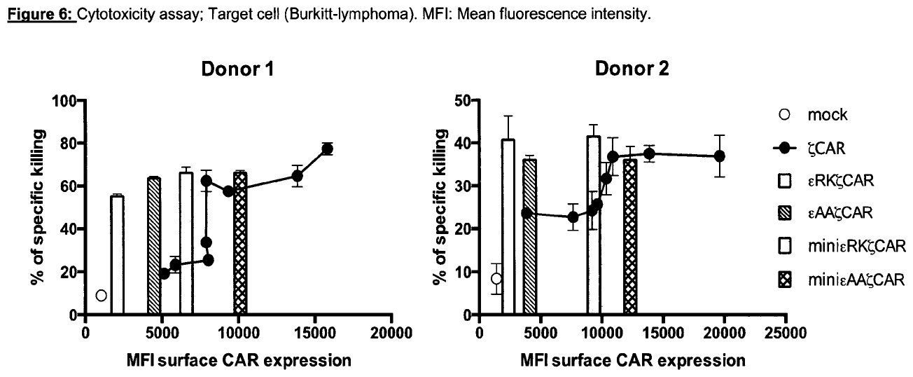

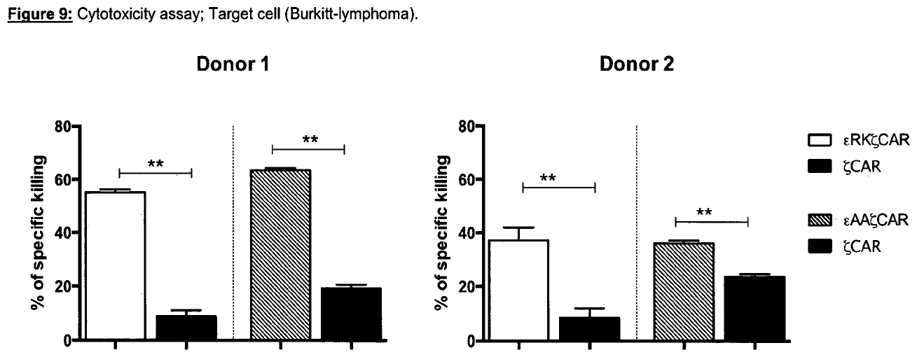

The anti-tumor activity (killing activity) of human T cells transduced with the lentiviral vector coding for the ζCAR at different multiplicities of infection (MOI) correlates with the level of surface expression of ζCAR. Donors were healthy volunteers. The target cells were DAUDI cells, which were derived from a Burkitt-lymphoma. Expanded human T cells transduced with the lentiviral vector were incubated with the target cells at 5:1 effector-to-target (E:T) ratio and incubated for 24 hours at 37° C. The specific anti-tumor activity was calculated as indicated in the methods section (Cytotoxicity assay). Comparison of the CARs according to the invention to the prior art CAR (ζCAR) at the same level of surface expression demonstrates that the embodiments of the present invention εRKζCAR, εAAζCAR and miniεRKζCAR have a much higher anti-tumoral activity. The results are shown in FIGS. 6, 9,10 and 13.

### Experiment 4:

T cell exhaustion is a major factor limiting anti-tumor responses. For this reason, we have checked up-regulation of the three exhaustion markers TIM3, LAG3 and PD1 upon expression of the ζCAR, miniεRKζCAR or miniεAAζCAR. To this end, 1.2×10⁵ CAR T cells expressing the indicated constructs were analyzed by flow cytometry for the surface expression of exhaustion markers. The analysis was performed 8 days after transduction. The following antibodies were using for the detection of exhaustion markers in CAR T cells: Alexa Fluor 647-labeled anti-human CD223 (LAG-3), PE/Cy7-labeled anti-human CD366 (Tim-3) and biotin-conjugated anti-human CD279 (PD-1). The results are shown in FIG. 14.

### Experiment 5:

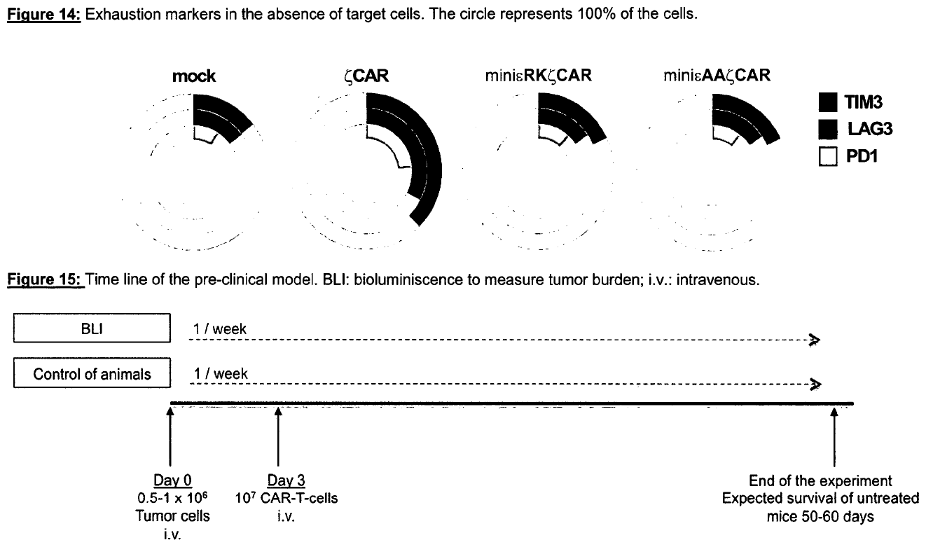

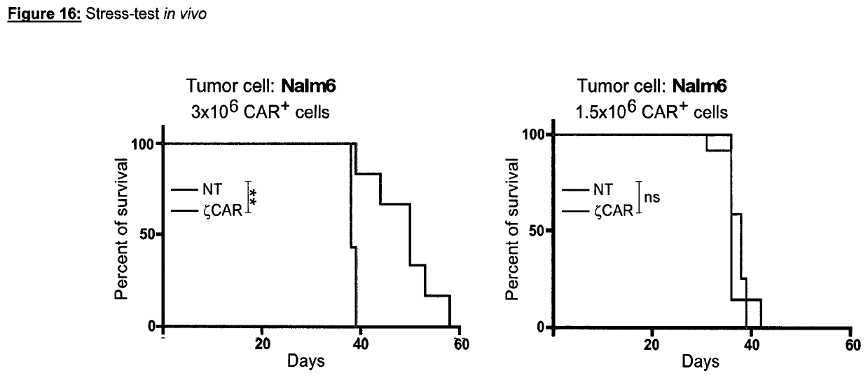

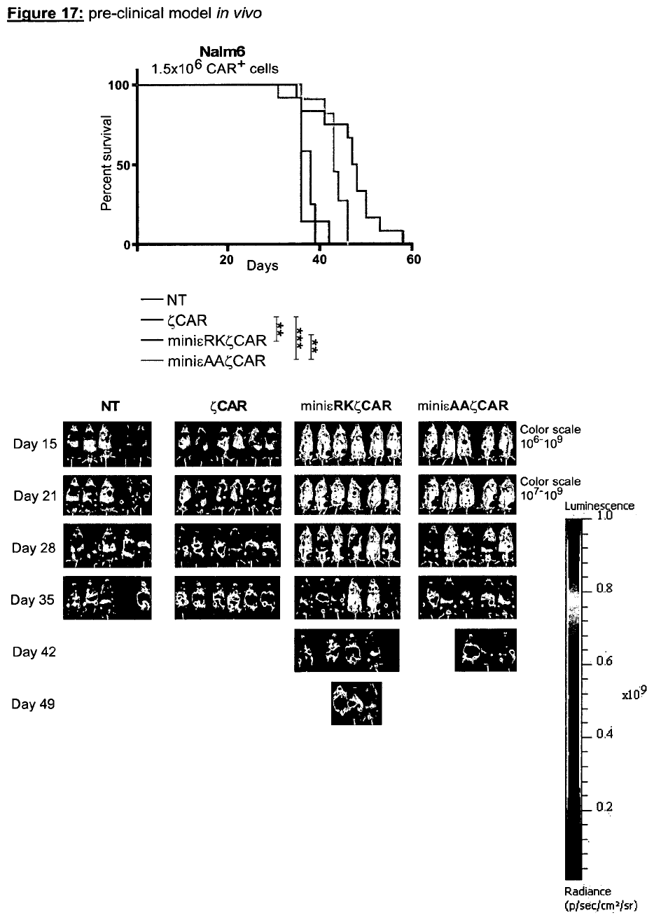

In order to test the anti-tumor activity of CARs in vivo, we performed a “stress test” in a pre-clinical mouse model using Nalm6 as previously described (Zhao et al., 2015). Briefly, Nalm6-tumor bearing mice were injected with CAR-T cell doses that are purposefully lowered to levels where well-established CAR therapy in vivo starts to fail. In our hands, this point was reached with a dose of 1.5×10⁶ ζCAR-positive cells. Mice were controlled weekly and the survival proportions plotted following the scheme shown in FIG. 15. The results are shown in FIG. 16.

### Experiment 6:

The “stress test” pre-clinical mouse model using Nalm6 was performed as in Experiment 5.Nalm6-tumor bearing mice were injected with 1.5×10⁶ CAR-positive cells. T cells expressing ζCAR,_miniεRKζCAR and miniεAAζCAR were used. Mice were controlled weekly and the survival proportions plotted. Mice were subjected to in vivo bioluminescence imaging (BLI) for firefly luciferase weekly. To this end, mice were injected i.p. with 200 mg/kg luciferin (Sigma, Germany) dissolved in distillated water. The mice were imaged in the bioluminescence camera 10 min after injection, with optimal settings for luminescent exposure. The results are shown in FIG. 17.

### Experiments 7 and 8: Duplication of the Binding Motif to the SH3 Domain of the SRC Family Kinase LCK in a TRuC™ Receptor Increases Activity

According to the present invention, the RK motif

(sequence RKGQRDLY, SEQ ID NO: 1)

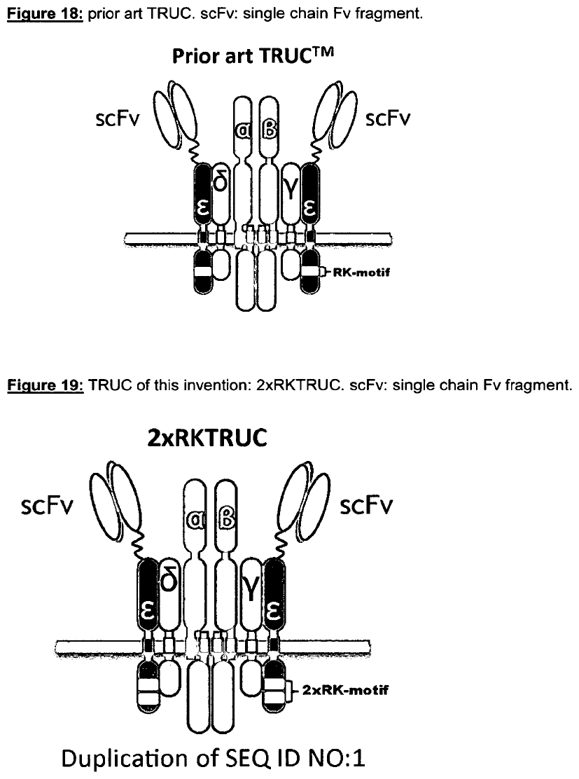

was introduced in a well-characterized TRuC (FIG. 18). The TRuC platform is a novel T cell therapy platform, which uses the complete TCR complex without the need for HLA matching. By conjugating the tumor antigen binder to the TCR complex, the TRuC construct recognizes surface antigens on tumor cells without the need for HLA and engage the complete TCR machinery to drive the totality of T cell functions required for potent, modulated and durable tumor killing.

The starting construct already express endogenously the RK motif

(sequence RKGQRDLY, SEQ ID NO: 1)

The impact of duplicating this motif to enhance LCK recruitment to the receptor on the anti-tumor functionality of T cells was analyzed. This approach serves as proof-of-principle of the applicability of adding a complementary TCR-derived activation domain include in preferred embodiments the amino acid sequence RKGQRDLY (SEQ ID NO:1).

Thus, a new TRuC was designed (designated as 2xRKTRuC FIG. 19), which is based on the well-characterized anti-CD19 εTRuC (FIG. 18) by duplicating the RK motif

(sequence RKGQRDLY, SEQ ID NO: 1)

right after the natural RK motif in CD3ε cytoplasmic tail. This chimeric receptor will be named 2xRKTRuC (FIG. 19).

This new receptor was used in the present invention and compared to the well-characterized anti-CD19εTRuC. The results are described in more detail in the following (FIGS. 18-21).

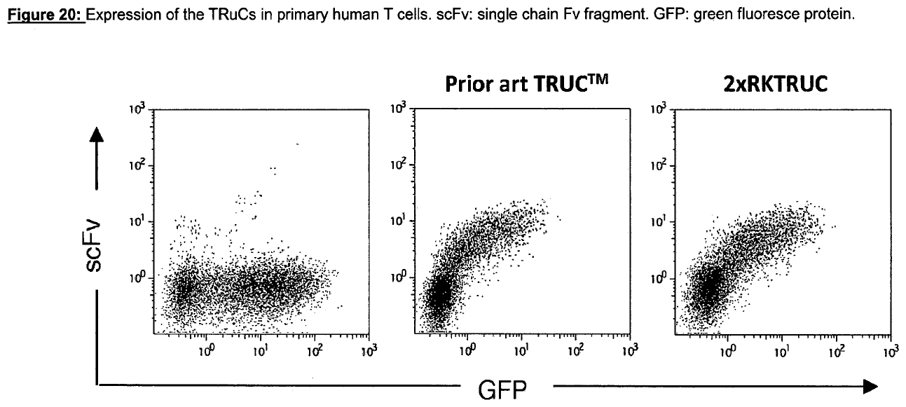

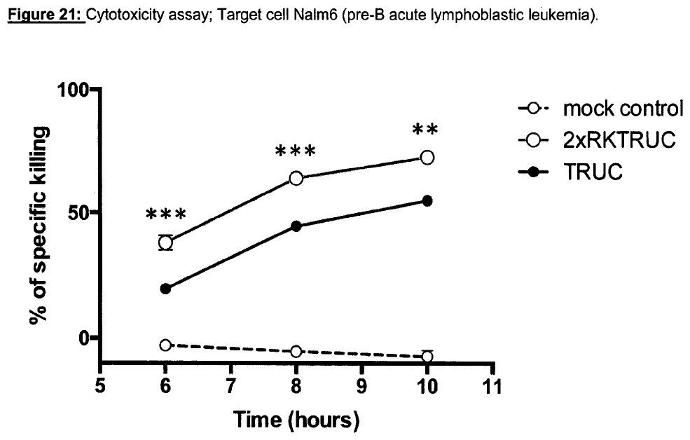

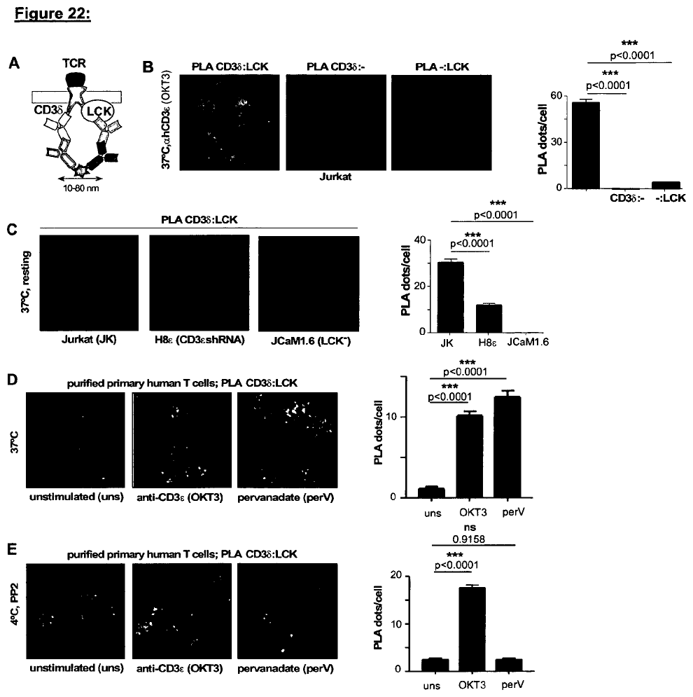

Experiment 7: Expanded human T cells from healthy donors were transduced with a lentiviral vector coding for the prior art TRuC or the novel 2xRKTRuC at a multiplicity of infection (MOI) of 5. The results are shown in FIG. 20.
Experiment 8: The anti-tumor activity (killing activity) of human T cells transduced with the lentiviral vector coding for the prior art TRuC or the novel 2xRKTRuC. Donors were healthy volunteers. The target cells were Nalm6 pre-B acute lymphoblastic leukemia. Expanded human T cells transduced with the lentiviral vector were incubated with the target cells at 1:1 effector-to-target (E:T) ratio and incubated at the indicated times at 37° C. The specific anti-tumor activity was calculated as indicated in the methods section (Cytotoxicity assay). Comparison of the 2xRKTRuC according to the invention to the prior art TRuC demonstrates that the embodiment of the present invention 2xRKTRuC has a higher anti-tumoral activity. The results are shown in FIG. 21 and demonstrate the improved efficiency of constructs according to the present invention.

1-15. (canceled)

16. A chimeric antigen receptor having two activatory domains which are TCR-derived, wherein said chimeric antigen receptor comprises at least the following components:
a) a peptidic structure capable of binding to a ligand, wherein said the peptide structure is an antigen binding structure;
b) an extracellular spacing structure;
c) a transmembrane domain;
d) none or at least one co-stimulatory domain;
e) at least one activatory domain which comprises an immune receptor tyrosine-based activation motif (“ITAM”) of the formula Yxx[I/L]x₆₋₉Yxx[L/I] that is phosphorylated by a SRC tyrosine kinase, and that does not contain the sequence RKGQRDLY (SEQ ID NO:1); and
f) a second activatory domain which is TCR-derived, wherein said second activatory domain consists of one or more repetitions the amino acid sequence RKGQRDLY (SEQ ID NO:1) which binds the lymphocyte specific SRC kinase (“LCK”) in a phosphorylation independent manner, said that the tyrosine (Y) is not phosphorylated.

17. The chimeric antigen receptor according to claim 16, characterized in that the second activatory domain, which is TCR-derived and consists of the amino acid sequence RKGQRDLY (SEQ ID NO:1), is derived from the cytoplasmatic tail of the CD3ε receptor.

18. The chimeric antigen receptor according to claim 16, characterized in that the sequence RKGQRDLY is capable of binding the lymphocyte specific Src kinase (LCK).

19. The chimeric antigen receptor according to claim 16, characterized in that said antigen binding structure is selected from among a single chain fragment (scFv), a nanobody, a naturally occurring ligand and an aptamer.

20. The chimeric antigen receptor according to claim 16, characterized in that said receptor includes said activatory domain that is TCR-derived and consists of the amino acid sequence RKGQRDLY (SEQ ID NO:1) but excludes any endoplasmatic reticulum retention signal naturally found in CD3ε amino acid sequence NQRRI (SEQ ID NO:3).

21. The chimeric antigen receptor according to claim 16, characterized in that said receptor components are spatially separated and not overlapping.

22. A vector comprising the genetic information coding for a chimeric antigen receptor according to claim 16.

23. The vector according to claim 22, characterized in that said vector is selected from the group consisting of: a lentiviral vector, a DNA vector, an RNA vector, a plasmid vector, a cosmid vector, a herpes virus vector, a measles virus vector, an adenoviral vector and a retrovirus.

24. A process for transfecting peripheral blood cells, wherein said process includes the steps of harvesting and concentrating peripheral blood cells transfected with the vector according to claim 22.

25. The process according to claim 24, wherein said process further comprises the steps of collecting T cells, isolating said T cells ex vivo and thereafter transfecting said isolated T cells with said vector.

26. The process according to claim 25, characterized in that the T cells transfected with said chimeric receptor are grown and expanded in vitro.

27. A peripheral blood cell comprising a chimeric antigen receptor according to claim 16, wherein said peripheral blood cell is obtained by a process that includes the steps of harvesting and concentrating peripheral blood cells transfected with a vector comprising the genetic information coding for a chimeric antigen receptor according to claim 16.

28. A method for treating or preventing cancer in a subject in need thereof, said method comprising the step of administering to said subject (1) a chimeric antigen receptor according to claim 16, (2) a vector comprising the genetic information coding for said chimeric antigen receptor, or (3) a peripheral blood cell comprising a chimeric antigen receptor according to claim 16 obtained by a process that includes the steps of harvesting and concentrating peripheral blood cells transfected with a vector comprising the genetic information coding for a chimeric antigen receptor according to claim 16.

29. A T cell comprising a chimeric antigen receptor according to claim 16 formulated for use in the prevention or treatment of cancer in a subject in need thereof.
